SEQUENCE LISTING (4 sequences)

SEQ ID NO: 1 - DNA, 1131 bp, Homo sapiens
  CDS (1)..(1131)
atgacaatcgctacaaaagtgaacacaaataaaaaggaccttgatacaatcaaggtaccggagcttccatcagtggcagctgtcaaagcagcaatccctgagcactgctttgtcaaggatccattgacttcaatttcatatcttatcaaggattacgtacttctcgctggtctctattttgcagttccatacattgagcattatctcggatggatcgggcttcttggatggtattgggcaatgggaattgttggatccgcattgttctgtgtgggtcatgactgtggacatggatcattctccgattatgaatggctcaatgatctttgtggacatttggctcatgctccaattcttgctccattctggccatggcaaaagtctcatagacaacatcatcaatacacatcccacgtggaaaaggataagggacatccatgggttactgaggaagactacaataatagaactgctattgagaagtatttcgctgtgattccaatttccggatggcttcgatggaatccaatctacaccatcgtcggtcttccagatggatctcatttctggccatggtcccggctcttcgagactactgaggatcgtgtcaagtgtgcagtttctggagttgcatgcgctatctgtgcttacattgcctttgtcctctgcgactattctgtctacacatttgtcaagtactactacattccacttctcttccaaggacttattctcgtcattatcacatatcttcaacatcagaatgaggatattgaggtctacgaagctgatgagtggggatttgtacgcggacaaacccaaactatcgacagacactggggattcggactcgacaacatcatgcacaacattaccaacggtcacgtcgcccatcacttcttcttcaccaaaatcccacattatcatctgttggaggcaactccagcaatcaagaaagctcttgaaccactgaaagacactcaatacggatacaaacgagaagtcaactataactggttcttcaagtatcttcactacaacgttaccctcgactatttgactcataaagcaaagggtgtcctgcaatacagaagtggagttgaggctgcaaaggctaagaaggctcaataa

SEQ ID NO: 2 - protein, 376 aa, Homo sapiens
MTIATKVNTNKKDLDTIKVPELPSVAAVKAAIPEHCFVKDPLTSISYLIKDYVLLAGLYFAVPYIEHYLGWIGLLGWYWAMGIVGSALFCVGHDCGHGSFSDYEWLNDLCGHLAHAPILAPFWPWQKSHRQHHQYTSHVEKDKGHPWVTEEDYNNRTAIEKYFAVIPISGWLRWNPIYTIVGLPDGSHFWPWSRLFETTEDRVKCAVSGVACAICAYIAFVLCDYSVYTFVKYYYIPLLFQGLILVIITYLQHQNEDIEVYEADEWGFVRGQTQTIDRHWGFGLDNIMHNITNGHVAHHFFFTKIPHYHLLEATPAIKKALEPLKDTQYGYKREVNYNWFFKYLHYNVTLDYLTHKAKGVLQYRSGVEAAKAKKAQ

SEQ ID NO: 3 - DNA, 15 bp, Artificial Sequence
  Description of Artificial Sequenceoligonucleotide
atgacaatcgctaca

SEQ ID NO: 4 - DNA, 15 bp, Artificial Sequence
  Description of Artificial Sequenceoligonucleotide
ttattgagccttctt

(19) United States
(12) Patent Application Publication
(10) Pub. No.: US 2003/0172398 A1
(43) Pub. Date: Sep. 11, 2003

(54) Novel delta-12 desaturase and methods of using it for synthesis of polyunsaturated fatty acids

(76) Inventor: John A. Browse, Palouse, WA (US)

Correspondence Address: KLARQUIST SPARKMAN CAMPBELL, LEIGH & WHINSTON, LLP, One World Trade Center, Suite 1600, 121 S.W. Salmon Street, Portland, OR, 97204-2988, US

(21) Appl. No.: 09/748,516

(22) Filed: Dec. 20, 2000

Related U.S. Application Data
(60) Provisional application No. 60/171,400, filed on Dec. 21, 1999.

(51) Int. Cl. C12N 15/82; A01H 5/00; C07H 21/04; C12N 9/02; C12N 5/04

(52) U.S. Cl. 800/281; 536/23.2; 435/189; 435/410; 800/288; 536/23.5

(57) ABSTRACT

Desaturase enzymes, and especially animal Δ¹²-desaturases, and the use of such enzymes to alter fatty acid saturation, especially fatty acid saturation in oilseeds, are disclosed. Also disclosed are nucleic acid sequences encoding animal Δ¹²-desaturase enzymes.

### ACKNOWLEDGMENT OF GOVERNMENT SUPPORT

This invention was made with government support from the United States Department of Agriculture, grant USDA-NRICGP 97-35301-4426, and the United States Department of Energy, grant DE-FG06-92ER20077. The government has certain rights in this invention.

### FIELD

The invention relates to animal desaturase enzymes and methods of using such enzymes to alter the saturation of fatty acids.

### INTRODUCTION

The unsaturation of fatty acids in glycerolipids is essential for the proper function of biological membranes. At physiological temperatures, polar glycerolipids that contain only saturated fatty acids cannot form the liquid-crystalline bilayer that is the fundamental structure of biological membranes (Stubbs and Smith, Biochim. Biophys. Acta, 779:89-137, 1984). The introduction of an appropriate number of unsaturated bonds into the fatty acids of membrane glycerolipids decreases the temperature for the transition from the solid to the liquid phase and provides membranes with the necessary fluidity (Russel, Trends Biochem. Sci., 9:108-112, 1984; and Hazel, Annu. Rev. Physiol., 57:19-42, 1995). Fluidity of the membrane is important for maintaining the barrier properties of the lipid bilayer and for the activation and function of certain membrane-bound enzymes (Houslay and Gordon, Curr. Top. Membr. Transp., 18:179-231, 1984; and Thompson, J. Bioenerg. Biomembr., 21:43-60, 1989). Many poikilothermic organisms respond to a decrease in temperature by desaturating the fatty acids of their membrane lipids (Cossins, Biochim. Biophys. Acta, 470:395-411, 1977; and Lee and Cossins, Biochim. Biophys. Acta, 1026:195-203, 1990). This homeoviscous adaptation (Sinensky, Proc. Natl. Acad. Sci. USA, 71:522-525, 1974; and McElhaney, Biomembranes, 12:249-276, 1984) improves the organisms' ability to maintain membrane fluidity over a broader temperature range and is believed to be an important component of cellular acclimation to temperature changes in poikilothermic organisms (Tiku, Science, 271:815-818, 1996).

In addition to their role in adaptation to low temperatures, membranes with unsaturated fatty acids also contribute to an organism's ability to adapt to other environmental stresses. For example, membrane lipid composition and membrane fluidity affects yeast tolerance to ethanol, with higher unsaturation correlating with higher ethanol tolerance (Alexandre et al., FEMS Microbiol. Lett., 124:17-22, 1994; Sajbidor and Grego, FEMS Microbiol. Lett., 93:13-16, 1992; Beavan et al., J. Ind. Microbiol., 128:1445-1447, 1982; and Del Castillo Agudo, Appl. Microbiol. Biotechnol., 37:647-651, 1992). However, the correlation is not exact (Swan and Watson, Can. J. Microbiol., 43:70-77, 1997; Guerzoni et al., Can. J. Microbiol., 43:569-476, 1997; and Swan and Watson, Can. J. Microbiol., 45:472-479, 1999), and it is likely that membrane fluidity is not the only factor to ethanol-stress resistance, since the synthesis of heat-shock proteins (Li, J. Cell Physiol., 115:116-122, 1983) and the synthesis of the disaccharide trehalose (Odumeru et al., J. Ind. Microbiol., 11:113-119, 1993) are both induced upon exposure of yeast to ethanol. There are many indications that ethanol and oxidative stress are connected to changes in membrane fluidity in mammals, particularly in fetal tissue (Henderson et al., Front. Biosci., 4:D541-D550, 1999), reproductive tissue (Zalata et al., Int. J. Androl., 21:154-162, 1998), and in human liver (French, Clin. Biochem., 22:41-49, 1989).

The ability of cells to modulate the degree of unsaturation in their membranes is mainly determined by the action of fatty acid desaturases (Kates et al., Biomembranes, 12:379-395, 1984; Murata and Wada, Biochem. J., 308:1-8, 1995; and Tocher et al., Prog. Lipid Res., 37: 73-117, 1998). Desaturase enzymes introduce unsaturated bonds at specific positions in their fatty acyl chain substrates. One classification of fatty acid desaturases is based on the moiety to which the hydrocarbon chains are acylated. Desaturases recognize substrates that are bound either to acyl carrier protein, to coenzyme A, or to lipid molecules (Murata and Wada, Biochem. J., 308:1-8, 1995; and Shanklin and Cahoon, Annu. Rev. Plant Physiol. Plant Mol. Biol., 49:611-641, 1998). Since desaturation reactions require one molecule of oxygen and two electrons for each reaction, desaturases also can be differentiated by the electron carrier that they require. While ferredoxin is the electron donor in the desaturation reactions catalyzed by acyl-ACP desaturases, by acyl-lipid desaturases of cyanobacteria, and by acyl-lipid desaturases in the plastids of plants (McKeon and Stumpf, J. Biol. Chem., 257:12141-12147, 1982; and Wada et al., J. Bacteriol., 175:544-547, 1993), the acyl lipid and acyl-CoA desaturases found in the endoplasmic reticulum of all eukaryotes and many bacteria use cytochrome b₅ as a donor (Jaworski, in The Biochemistry of Plants (Stumpf et al., Eds.), Academic Press, Orlando, Fla., Vol. 9:159-174, 1987; Macartney et al., in Temperature Adaptation of Biological Membranes (Cossins, ed.), Portland Press, London, pp. 129-139, 1994; and Jaworski and Stumpf, Arch. Biochem. Biophys., 162:158-165, 1974). Desaturase enzymes also show considerable selectivity both for the chain length of the substrate and for the location of existing double bonds in the fatty acyl chain (Shanklin and Cahoon, Annu. Rev. Plant Physiol. Plant Mol. Biol., 49:611-641, 1998).

Purification and activity of fatty acid desaturases have been limited by their requirement for membrane association. One of the most fruitful approaches to examining desaturase activity has been mutational analysis. Isolation of mutants in cyanobacteria and Arabidopsis thaliana with altered fatty acid compositions has permitted the isolation of genes encoding most of the transmembrane desaturases present in these organisms (Browse et al., Science, 227:763-765, 1985; and Browse and Somerville, in Arabidopsis (Meyerowitz and Somerville, eds.), Cold Spring Harbor Laboratory Press, Plainview, N.Y., pp. 881-912, 1994). Sequence analysis of these desaturases has facilitated the cloning of a number of other desaturase genes from plants (Tocher et al., Prog. Lipid Res., 37:73-117, 1998; and Sayanova et al., Proc. Natl. Acad. Sci. USA, 94:4211-4216, 1997), bacteria (Aguilar et al., J. Bacteriol., 180:2194-2200, 1998), protists (Nakashima et al., Biochem. J., 317(Pt 1):29-34, 1996), nematodes (Spychalla et al., Proc. Natl. Acad. Sci. USA, 94:1142-1147, 1997; Watts and Browse, Arch. Biochem. Biophys., 362:175-182, 1999; and Napier et al., Biochem. J., 330:611-614, 1998), and mammals (Cho et al., J. Biol. Chem., 274:471-477, 1999; and Aki et al., Biochem. Biophys. Res. Commun., 255:575-579, 1999).

While most eukaryotic organisms, including mammals, can introduce a double bond into an 18-carbon fatty acid at the Δ⁹ position, mammals are incapable of inserting double bonds at the Δ¹² or Δ¹⁵ positions. For this reason, linoleate (18:2 Δ^{9,12}) and linolenate (18:3 Δ^{9,12,15}) must be obtained from the diet, and are termed “essential” fatty acids. These dietary fatty acids come predominantly from plant sources, since flowering plants readily desaturate at both the Δ¹² and Δ¹⁵ positions. Certain animals, however, including some insects and nematodes, can synthesize de novo all their component fatty acids including linoleate and linolenate. The nematode Caenorhabditis elegans can synthesize de novo a broad range of polyunsaturated fatty acids including arachidonic acid and eicosapentaenoic acids, an accomplishment not shared by either mammals or flowering plants (Hutzell and Krusberg, Comp. Biochem. Physiol., 73B:1173-1178, 1982; and Tanaka et al., Lipids, 31:1173-1178, 1996).

The Arabidopsis Δ¹²-desaturase has been described (Okuley et al., Plant Cell, 6:147-158, 1994), and a number of similar sequences have been obtained from other plants (Tocher et al., Prog. Lipid Res., 37:73-117, 1998). The activity of animal Δ¹²-desaturation has been studied in insects (Cripps et al., Arch. Biochem. Biophys., 278:46-51, 1990; and Borgeson et al., Biochim. Biophys. Acta, 1047:135-140, 1990). Biochemical characterization of insect Δ¹²-desaturases suggests that there may be differences between substrates used by plants and animals. Available evidence indicates that, unlike the plant enzymes, the cricket Δ¹²-desaturase activity uses acyl-CoA as substrates (Borgeson et al., Biochim. Biophys. Acta, 1047:135-140, 1990). No gene encoding an animal Δ¹²-desaturase has previously been isolated.

Acquisition of the gene encoding an animal Δ¹²-desaturase would represent an important advance in efforts to alter and control saturation of fatty acids.

### SUMMARY

The invention provides an isolated fat-2 cDNA from Caenorhabditis elegans that is shown to affect fatty acid saturation when transformed into host cells, and the FAT-2 protein encoded by this nucleic acid. This animal Δ¹²-desaturase provides surprisingly high desaturation activity when compared to known plant Δ¹²-desaturases.

The novel animal Δ¹²-desaturase enzymes of this invention may be cloned and expressed in the cells of various organisms, including plants, to produce polyunsaturated fatty acids. Expression of such polyunsaturated fatty acids enhances the nutritional qualities of such organisms. For instance, oil-seed plants may be engineered to incorporate a Δ¹²-desaturase of the invention. Such oil-seed plants would produce seed-oil rich in polyunsaturated fatty acids. Such fatty acids could be incorporated usefully into infant formula, foods of all kinds, dietary supplements, and nutriceutical and pharmaceutical formulations.

The invention also provides proteins differing from these proteins by one or more conservative amino acid substitutions. Also provided are proteins that exhibit “substantial similarity” (defined in the “Definitions” section) with these Δ¹²-desaturase proteins.

The invention provides isolated novel nucleic acids that encode the above-mentioned proteins, recombinant nucleic acids that include such nucleic acids and cells, plants, and other organisms containing such recombinant nucleic acids. Appropriate plants include oil palm, sunflower, safflower, rapeseed, canola, soy, peanut, cotton, corn, rice, Arabidopsis, mustard, wheat, barley, potato, tomato, yam, apple, and pear plants.

The novel Δ¹²-desaturase proteins can be used to produce polyunsaturated fatty acids, such as 16:2 and 18:2 fatty acids.

The scope of the invention also includes portions of nucleic acids encoding the novel Δ¹²-desaturase enzymes, portions of nucleic acids that encode polypeptides substantially similar to these novel enzymes, and portions of nucleic acids that encode polypeptides that differ from the inventive proteins by one or more conservative amino acid substitutions. Such portions of nucleic acids may be used, for instance, as primers and probes for research and diagnostic purposes. Research applications for such probes and primers include the identification and cloning of related Δ¹²-desaturases in other organisms including both eukaryotes and prokaryotes.

The invention also includes methods that utilize the Δ¹²-desaturase enzymes of the invention. An example of this embodiment is a yeast or plant cell that carries genes for a Δ¹²-desaturase of the invention and that, by virtue of this desaturase, is able to produce polyunsaturated fatty acids.

The foregoing and other objects, features, and advantages of the invention will become more apparent from the following detailed description of several embodiments, which proceeds with reference to the accompanying sequence listing and figures.

### SEQUENCE LISTING

The nucleic acid and amino acid sequences listed in the accompanying sequence listing are shown using standard letter abbreviations for nucleotide bases, and the three-letter code for amino acids. Only one strand of each nucleic acid sequence is shown, but the complementary strand is understood to be included by any reference to the displayed strand.

SEQ ID NO: 1 is the nucleotide and amino acid sequence of C. elegans fat-2 cDNA.

SEQ ID NO: 2 is the amino acid sequence of C. elegans FAT-2 protein.

SEQ ID NOs: 3 and 4 are oligonucleotide primers that can be used to amplify the fat-2 cDNA.

### FIGURES

FIG. 1 shows a comparative sequence alignment of the deduced amino acid sequence of C. elegans fat-2 and fat-1 genes (fat2 and fat1), and Arabidopsis thaliana FAD2 and FAD3 (fad2 and fad3). Amino acid identities are shaded black, and conserved residues are shaded gray. The three conserved histidine-rich motifs are indicated below the relevant section of aligned sequences.

FIGS. 2(A)-2(D) show gas chromatography traces of wild-type yeast transformed with the empty vector (FIG. 2(A)) and transgenic yeast transformed with fat-2 (FIG. 2(B)). Fatty acid methyl esters (FAMEs) of total fatty acids were identified as follows: (1) 16:0, (2) 16:1, (3) 16:2, (4) 18:0, (5) 18:1, (6) 18:2. Also shown are the mass spectra of polyunsaturated fatty acids from transgenic yeast expressing FAT-2: (C) 16:2, (D) 18:2.

FIG. 3 shows the relative fluidity of yeast membranes containing the fluoroprobe diphenyhexatriene (DPH), measured as fluorescence polarization (P) at different temperatures. Fluorescence polarization measurements were carried out on a spectrofluorometer in a T-format. Excitation was provided by light at 360 nm with a band-pass of 2 nm. Fluorescence was monitored with cut off filters at 470 nm. The standard deviation is less than 10% of P values.

□: Wild-type strain.

M: Transgenic strain.

FIGS. 4(A)-4(B) show the results of stress-tolerance tests of fat-2 transgenic yeast and wildtype controls, as the percentage of survival of yeast cells. Cells were grown until early log phase, washed with 67 mM phosphate buffer and resuspended in the same buffer. The cells they were treated subsequently with either 10% ethanol (v/v) (FIG. 4(A)) or 3 mM hydrogen peroxide (FIG. 4(B)) for 8 hours. The error bars indicate the standard deviation of three measurements.

□: Untreated control strain.

*: Untreated fat-2 yeast.

Δ: Treated control strain.

◯: Treated fat-2 yeast.

FIG. 5(A) shows the relative location of C. elegans Δ⁶ (fat-3), Δ⁵ (fat-4), ω³ (fat-1), and Δ¹² (fat-2) desaturase genes on chromosome IV. Approximate map locations are 3.03 for fat-4, 3.08 for fat-3, 5.52 for fat-2; fat-2 and fat-1 are separated by approximately 5.3 kb.

FIG. 5(B) shows the structure of fat-1 and fat-2 on their respective YAC (Y67H2) and cosmid (W02A2). Introns are shaded.

### DETAILED DESCRIPTION

I. Definitions

The following definitions and methods are provided to better define the present invention and to guide those of ordinary skill in the art in the practice of the present invention. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1991; and Lewin, Genes VI, Oxford University Press: New York, 1997. The nomenclature for DNA bases as set forth at 37 C.F.R. § 1.822 is used. The standard one- and three-letter nomenclature for amino acid residues is used.

cDNA (complementary DNA): A “cDNA” is a piece of DNA lacking internal, non-coding segments (introns) and regulatory sequences that determine transcription. cDNA is synthesized in the laboratory by reverse transcription from messenger RNA extracted from cells.

Desaturase: A desaturase is an enzyme that promotes the formation of carbon-carbon double bonds in a hydrocarbon molecule.

Desaturase activity may be demonstrated by assays in which a preparation containing a putative desaturase enzyme is incubated with a suitable substrate fatty acid and analyzed for conversion of the substrate to a predicted fatty acid product. Alternatively, a DNA sequence proposed to encode a desaturase protein may be incorporated into a suitable vector construct and thereby expressed in cells of a type that do not normally have an ability to desaturate a particular fatty acid substrate. Activity of the desaturase enzyme encoded by the DNA sequence then can be demonstrated by supplying a suitable form of substrate fatty acid to cells transformed with a vector containing the desaturase-encoding DNA sequence and to suitable control cells (for example, transformed with the empty vector alone). In such an experiment, detection of the predicted fatty acid product in cells containing the desaturase-encoding DNA sequence and not in control cells establishes the desaturase activity. Examples of this type of assay have been described in, for example, Lee et al., Science, 280:915-918, 1998; Napier et al., Biochem. J., 330:611-614, 1998; and Michaelson et al., J. Biol. Chem., 273:19055-19059, 1998, incorporated herein by reference.

Δ¹²-desaturase activity may be assayed by these techniques using, for example, 18:1Δ⁹ as substrate and detecting 18:2Δ^{9,12} as the product, as described herein. Other potential substrates for use in Δ⁵-activity assays include (but are not limited to) 16:1Δ⁹ (yielding 16:2Δ^{9,12} as the product) and 20:1Δ⁹ (yielding 21:2Δ^{9,12} as the product).

DNA construct: The term “DNA construct” is intended to indicate any nucleic acid molecule of cDNA, genomic DNA, synthetic DNA, or RNA origin. The term “construct” is intended to indicate a nucleic acid segment that may be single- or double-stranded, and that may be based on a complete or partial naturally occurring nucleotide sequence encoding one or more of the transacylase genes of the present invention. It is understood that such nucleotide sequences include intentionally manipulated nucleotide sequences, e.g., subjected to site-directed mutagenesis, and sequences that are degenerate as a result of the genetic code. All degenerate nucleotide sequences are included within the scope of the invention so long as the transacylase encoded by the nucleotide sequence maintains transacylase activity as described below.

Homologs: “Homologs” are two nucleotide sequences that share a common ancestral sequence and diverged when a species carrying that ancestral sequence split into two species.

Isolated: An “isolated” biological component (such as a nucleic acid or protein or organelle) is a component that has been substantially separated or purified away from other biological components in the cell of the organism in which the component naturally occurs, i.e., other chromosomal and extra-chromosomal DNA, RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

Mammal: This term includes both humans and non-human mammals. Similarly, the term “patient” includes both humans and veterinary subjects.

Operably linked: A first nucleic acid sequence is “operably linked” with a second nucleic acid sequence whenever the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

ORF (open reading frame): An “ORF” is a series of nucleotide triplets (codons) coding for amino acids without any termination codons. These sequences are usually translatable into respective polypeptides.

Orthologs: An “ortholog” is a gene that encodes a protein that displays a function that is similar to a gene derived from a different species.

Primers: Short nucleic acids, preferably DNA oligonucleotides 10 nucleotides or more in length, that are annealable to a complementary target DNA strand by nucleic acid hybridization to form a hybrid between the primer and the target DNA strand, then extendable along the target DNA strand by a DNA polymerase enzyme. Primer pairs can be used for amplification of a nucleic acid sequence, e.g., by the polymerase chain reaction (PCR) or other nucleic-acid amplification methods known in the art.

Probes and primers as used in the present invention typically comprise at least 15 contiguous nucleotides. In order to enhance specificity, longer probes and primers may also be employed, such as probes and primers that comprise at least 20, 30, 40, 50, 60, 70, 80, 90, 100, or 150 consecutive nucleotides of the disclosed nucleic acid sequences.

Alternatively, such probes and primers may comprise at least 15, 20, 30, 40, 50, 60, 70, 80, 90, 100, or 150 consecutive nucleotides that share a defined level of sequence identity with one of the disclosed sequences, for instance, at least a 50%, 60%, 70%, 80%, 90%, or 95% sequence identity.

Alternatively, such probes and primers may be nucleotide molecules that hybridize under specific conditions and remain hybridized under specific wash conditions such as those provided below. These conditions can be used to identifying variants of the desaturases. Nucleic acid molecules that are derived from the desaturase cDNA and gene sequences include molecules that hybridize under various conditions to the disclosed desaturase nucleic acid molecules, or fragments thereof. Generally, hybridization conditions are classified into categories, for example very high stringency, high stringency, and low stringency. The conditions for probes that are about 600 base pairs or more in length are provided below in three corresponding categories.

Very High Stringency (detects sequences
that share 90% sequence identity)

| | | | | | |
|---|---|---|---|---|---|
| Hybridization | in | SSC | at | 65° C. | 16 hours |
| Wash twice | in | SSC | at | room temp. | 15 minutes each |
| Wash twice | in | SSC | at | 65° C. | 20 minutes each |

High Stringency (detects sequences
that share 80% sequence identity or greater)

| | | | | | |
|---|---|---|---|---|---|
| Hybridization | in | SSC | at | 65° C.-70° C. | 16-20 hours |
| Wash twice | in | SSC | at | room temp. | 5-20 minutes each |
| Wash twice | in | SSC | at | 55° C.-70° C. | 30 minutes each |

Low Stringency (detects sequences
that share greater than 50% sequence identity)

| | | | | | |
|---|---|---|---|---|---|
| Hybridization | in | SSC | at | room | 16-20 hours |
| | | | | temp. −55° C. | |
| Wash at | in | SSC | at | room | 20-30 minutes each |
| least twice | | | | temp. −55° C. | |

Methods for preparing and using probes and primers are described in the references, for example, Sambrook et al., 1989; Ausubel et al. (eds.) Current Protocols in Molecular Biology, John Wiley & Sons, New York (with periodic updates), 1998; and Innis et al., PCR Protocols, A Guide to Methods and Applications, Academic Press, Inc., San Diego, Calif. 1990. PCR primer pairs can be derived from a known sequence, for example, by using computer programs intended for that purpose such as Primer™ (Version 0.5, 1991, Whitehead Institute for Biomedical Research, Cambridge, Mass.).

Probe: An isolated nucleic acid attached to a detectable label or reporter molecule. Typical labels include radioactive isotopes, ligands, chemiluminescent agents, and enzymes.

Purified: The term “purified” does not require absolute purity; rather, it is intended as a relative term. Thus, for example, a purified enzyme or nucleic acid preparation is one in which the subject protein or nucleotide, respectively, is at a higher concentration than the protein or nucleotide would be in its natural environment within an organism. For example, a preparation of an enzyme can be considered as purified if the enzyme content in the preparation represents at least 50% of the total protein content of the preparation.

Recombinant: A “recombinant” nucleic acid is one having a sequence that is not naturally occurring in the organism in which it is expressed, or has a sequence made by an artificial combination of two otherwise-separated, shorter sequences. This artificial combination is often accomplished by chemical synthesis or, more commonly, by the artificial manipulation of isolated segments of nucleic acids, e.g., by genetic engineering techniques. “Recombinant” is also used to describe nucleic acid molecules that have been artificially manipulated, but contain the same control sequences and coding regions that are found in the organism from which the gene was isolated.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in the following: Smith & Waterman, Adv. Appl. Math., 2:482, 1981; Needleman & Wunsch, J. Mol. Biol., 48:443, 1970; Pearson & Lipman, Proc. Natl. Acad. Sci. USA, 85:2444, 1988; Higgins & Sharp, Gene, 73:237-244, 1988; Higgins & Sharp, CABIOS, 5:151-153, 1989; Corpet et al., Nucleic Acids Research, 16:10881-10890, 1988; Huang, et al., Co. Applications in the Biosciences, 8:155-165, 1992; and Pearson et al., Methods in Molecular Biology, 24:307-331, 1994. Altschul et al., J. Mol. Biol., 215:403-410, 1990, presents a detailed consideration of sequence alignment methods and homology calculations.

The National Center for Biotechnology Information (NCBI) Basic Local Alignment Search Tool (BLAST™) (Altschul et al., J. Mol. Biol. 215:403-410, 1990 is available from several sources, including the NCBI, Bethesda, Md., and on the Internet at the NCBI website, for use in connection with the sequence analysis programs blastp, blastn, blastx, tblastn and tblastx. A description of how to determine sequence identity using this program is available at the web site. As used herein, sequence identity is commonly determined with the BLAST™ software set to default parameters. For instance, blastn (version 2.0) software may be used to determine sequence identity between two nucleic acid sequences using default parameters (expect=10, matrix=BLOSUM62, filter=DUST (Hancock and Armstrong, Comput. Appl. Biosci. 10:67-70, 1994), gap existence cost=11, per residue gap cost=1, and lambda ratio=0.85). For comparison of two polypeptides, blastp (version 2.0) software may be used with default parameters (expect 10, filter=SEG (Wootton and Federhen, Computers in Chemistry 17:149-163, 1993), matrix=BLOSUM62, gap existence cost=11, per residue gap cost=1, lambda=0.85).

When aligning short peptides (fewer than about 30 amino acids), the alignment should be performed using the Blast 2 sequences function, employing the PAM30 matrix set to default parameters (open gap 9, extension gap 1 penalties).

An alternative alignment tool is the ALIGN™ Global Optimal Alignment tool (version 3.0) available from Biology Workbench at http://biology.ncsa.uiuc.edu. This tool may be used with settings set to default parameters to align two known sequences. References for this tool include Meyers and Miller, CABIOS, 4:11-17, 1989.

Specific binding agent: An agent that binds substantially only to a defined target. Thus a FAT-2 protein-specific binding agent binds substantially only the FAT-2 protein. As used herein, the term “FAT-2 protein specific binding agent” includes anti-FAT-2 protein antibodies and other agents (such as soluble receptors) that bind substantially only to the FAT-2 protein.

Anti-FAT-2 protein antibodies may be produced using standard procedures described in a number of texts, including Harlow and Lane, Antibodies, A Laboratory Manual, Cold Spring Harbor Laboratory, New York, 1988. The determination that a particular agent binds substantially only to the FAT-2 protein may readily be made by using or adapting routine procedures. One suitable in vitro assay makes use of the Western blotting procedure (described in many standard texts, including Harlow and Lane, Antibodies, A Laboratory Manual, Cold Spring Harbor Laboratory, New York, 1988. Western blotting may be used to determine that a given FAT-2 protein binding agent, such as an anti-FAT-2 protein monoclonal antibody, binds substantially only to the FAT-2 protein.

Shorter fragments of antibodies can also serve as specific binding agents. For instance, Fabs, Fvs, and single-chain Fvs (SCFvs) that bind to FAT-2 would be FAT-2-specific binding agents. These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single-chain antibody (“SCA”), a genetically engineered molecule containing the variable region of the light chain, the variable region of the heavy chain, linked by a suitable polypeptide linker as a genetically fused single-chain molecule. Methods for making these fragments are routine.

Substantial similarity: A first nucleic acid is “substantially similar” to a second nucleic acid if, when optimally aligned (with appropriate nucleotide deletions or gap insertions) with the other nucleic acid (or its complementary strand), there is nucleotide-sequence identity in at least about, for example, 50%, 75%, 80%, 85%, 90% or 95% of the nucleotide bases. Sequence similarity can be determined by comparing the nucleotide sequences of two nucleic acids using the BLAST™ sequence analysis software (blastn) available from The National Center for Biotechnology Information. Such comparisons may be made using the software set to default settings (expect=10, filter=default, descriptions=500 pairwise, alignments=500, alignment view=standard, gap existence cost=11, per residue existence=1, per residue gap cost=0.85). Similarly, a first polypeptide is substantially similar to a second polypeptide if they show sequence identity of at least about 75%-90% or greater when optimally aligned and compared using BLAST software (blastp) using default settings.

Transformed: A “transformed” cell is a cell into which a nucleic acid molecule has been introduced by molecular biology techniques. As used herein, the term “transformation” encompasses all techniques by which a nucleic acid molecule might be introduced into such a cell, including transfection with a viral vector, transformation with a plasmid vector, and introduction of naked DNA by electroporation, lipofection, and particle gun acceleration.

Vector: A “vector” is a nucleic acid molecule as introduced into a host cell, thereby producing a transformed host cell. A vector may include nucleic acid sequences, such as an origin of replication, that permit the vector to replicate in a host cell. A vector may also include one or more screenable markers, selectable markers, or reporter genes and other genetic elements known in the art.

II. Isolation of a C. elegans Δ¹²-Desaturase

This invention reports the isolation of a cDNA that encodes an animal fatty acid Δ¹²-desaturase (SEQ ID NO: 1). The corresponding gene is termed fat-2. The FAT-2 protein (SEQ ID NO: 2) is the first representative of the animal Δ¹²-desaturase class. FAT-2 is able to act on both 16:1Δ⁹ and 18:1Δ⁹ to produce 16:2Δ^{9,12} and 18:2Δ^{9,12} polyunsaturated fatty acids, respectively. Surprisingly, FAT-2 provides a substantially greater accumulation of 16:2 and 18:2 fatty acids compared with the published results on the expression of FAD2 in yeast.

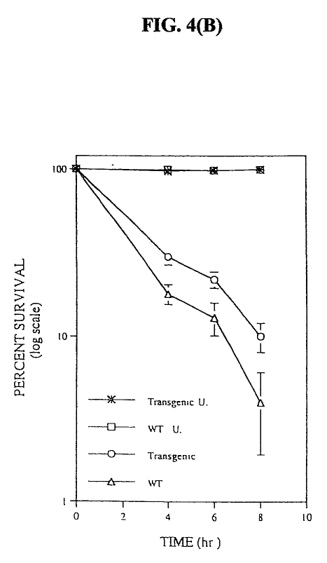

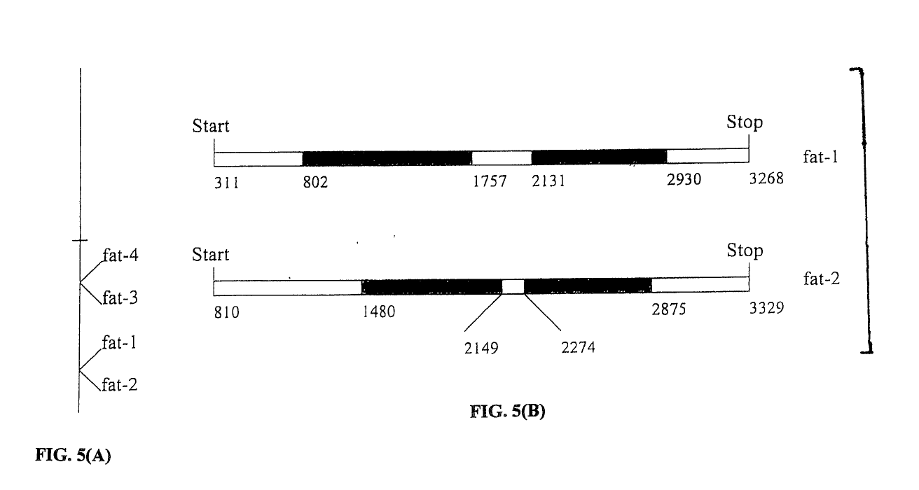

The FAT-2 protein corresponds to a predicted protein, W02A2.1 (GenPept accession number CAB05394; see FIG. 5B), identified by the C. elegans genome-sequencing project. Examination of the nematode genome reveals that all four cloned C. elegans fatty acid desaturase genes, fat-1 (Δ³), fat-3 (Δ⁶), fat-4 (Δ⁵), and fat-2, lie on the right arm of chromosome 4 (LGIV) (FIG. 5(A)). The fat-3 and fat-4 genes are transcribed in the same 5′→3′ orientation with only 0.85 kb separating them (Okuley et al., Plant Cell, 6:147-158, 1994). Their amino acid sequences are 45% identical and they share two intron/exon boundaries, indicating that these two desaturase activities could have arisen from an ancient gene-duplication event. The fat-1 and fat-2 genes are also transcribed in the same 5′→3′ orientation, have similar structures of three exons and two introns, and share 51% amino acid identity. However, they are separated by approximately 5.3 kb of DNA. It is possible that these two genes also arose from an ancient gene-duplication event.

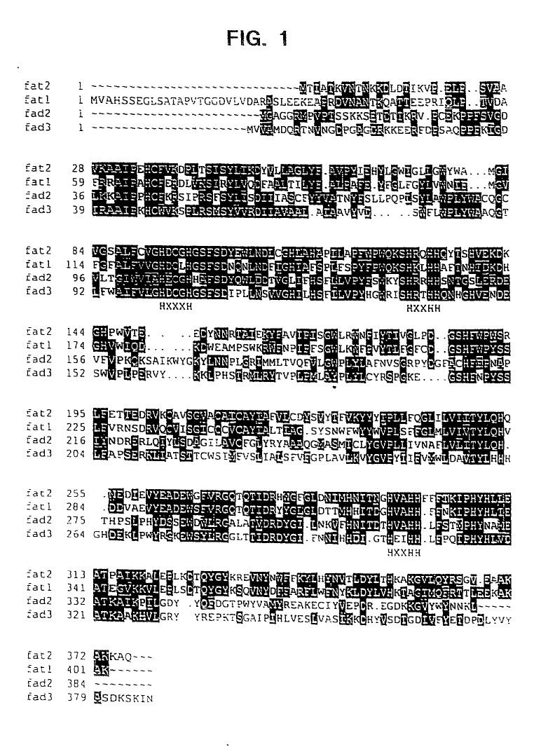

The predicted FAT-2 protein (SEQ ID NO: 2) includes three histidine-rich sequences that are highly conserved among membrane-bound desaturases and have been shown to be necessary for enzyme function in other Δ¹²-desaturases (Shanklin et al., Biochem., 33:12787-12794, 1994). It is believed that these residues coordinate the diiron-oxo structure at the active site of the desaturases. The FAT-2 protein contains two significant hydrophobic stretches, each long enough to span the membrane twice (residues 69 to 117, and 230 to 281, in FIG. 1). In FAT-2, the position and length of these stretches relative to the conserved histidine boxes (FIG. 1) are similar to other membrane-bound desaturases. Thus, the FAT-2 protein conforms to the model proposed by Stukey et al., J. Biol. Chem., 265:20144-20149, 1990, in which the peptide chain spans the membrane four times and exposes the three histidine clusters on the cytoplasmic side of the endoplasmic reticulum. Unlike the native yeast Δ⁹-desaturase, which has a cytochrome-like domain at its carboxyl terminus, the C. elegans FAT-2 may interact with a separate cytochrome b₅ to achieve its activity both in the nematode and in transgenic yeast.

Transformed yeast expressing the FAT-2 enzyme contained high levels of polyunsaturated fatty acids. Physiological studies of these transformed yeast (see below) demonstrate both that their membrane fluidity and growth characteristics are altered, and that they have increased resistance to ethanol and hydrogen peroxide stress. The C. elegans Δ¹²-desaturase gene fat-2 is responsible for conveying these novel characteristics on the transformed yeast.

A. Materials and Methods

Cloning and sequencing of a fat-2 cDNA

The NCBI's Expressed Sequence Tag (EST) database was searched with BLAST (Altschul et al., J. Mol. Biol., 215:403-410, 1990), using the peptide sequences of the Arabidopsis thaliana FAD2 (GenBank accession L26296), FAD6 (U09503), and FAD7 (D14007) fatty acid desaturases as queries. Two partial cDNA clones identified by these searches, CEL20a7 and CEL18f3, were obtained from the C. elegans Genome Sequencing Center at Washington University School of Medicine in St. Louis. The cDNA was labeled with [α-³²P] dCTP using a random priming kit (Prime-a-Gene™; Promega, Madison, Wis.), and the labeled probe was used to screen a C. elegans mixed-stage lambda phage Uni-ZAP XR library (Stratagene, La Jolla, Calif.). Positive clones were excised from the phage vector according to the manufacturer's protocol to yield pBluescript™ plasmids. The clone with the longest insert, pCM1 8, was sequenced in both directions using dye-termination sequencing technology (Applied Biosystems™, Foster City, Calif.). Analysis of the sequences was carried out using programs available in the Genetics Computer Group package (Devereux et al., Nucleic Acids Res., 12:387-395, 1984), except for analysis of transmembrane domains, which was conducted with the SOSUI server at the Tokyo University of Agriculture and Technology.

Yeast expression

The plasmid pCM18 was restricted with EcoRI and XhoI to excise the cDNA, and the isolated fragment was ligated into the episomal yeast expression vector pMK195 (Overvoorde et al., Plant Cell, 8:271-280, 1996) that had been digested with the same enzymes. Directional cloning of the cDNA into this vector provided for expression of the FAT-2 protein under the control of the constitutive ADH1 promoter. The resulting construct, pMK195-fat-2, was introduced into Saccharomyces cerevisiae strain YRP685 (MATa, leu2, lys2, his4, trp1, ura3) using the lithium acetate procedure (Ausubel et al. (eds.), Current Protocols in Molecular Biology, John Wiley & Sons, New York, 13.7.1-13.12.2, 1994). Transformed cells were grown in a complete minimal medium supplemented with 2% glucose but lacking uracil (since pMK195 encodes ura prototrophy).

Lipid Analysis

Methods for extraction and separation of lipids and for the analysis of fatty acid methyl esters (FAMEs) have been described (Miquel and Browse, J. Biol. Chem., 267:1502-1509, 1992). Briefly, cells were grown overnight in selective medium in the presence of glucose. One milliliter of the culture was centrifuged and cells were resuspended in 2.5% sulfuric acid in methanol. The mixture was incubated at 80° C. for one hour and the resulting fatty acid methyl esters were extracted in hexane. Analysis was performed by gas chromatography-mass spectrometry (GC-MS). GC-MS analysis was carried out on a 30 m×0.2 mm AT1000 column (Alltech Associates, Deerfield, Ill.) in a HP6890 instrument (Hewlett-Packard, Palo Alto, Calif.). Oven temperature at injection was 150° C., which was increased at 5° C./minute to 230° C., then held at 230° C. for 10 minutes. Novel fatty acids were identified by comparison of their retention times and mass spectra with authentic 16:2 and 18:2 fatty acids (NuChek-Prep, Elysian, Minn.).

Microsome Preparation

Microsomes were prepared by modification of an established protocol (Bonitz et al., J. Biol. Chem., 255:11927-11941, 1980). Briefly, the control and experimental yeast strains were grown at 28° C. to late log phase, and then pelleted by centrifugation at 5,000×g for 5 minutes. After discarding the supernatant, the cells were washed once with 1.2 M sorbitol, then suspended in a protoplasting solution of 30 mg Zymolyase 20T (Sigma, St. Louis, Ill.), 18 mL 2M sorbitol, 4.5 mL 0.5M KHPO₄ pH 7.5, 0.75 mL β-mercaptoethanol, 12 μL 0.5 M EDTA, and 6.7 mL water. The suspension was incubated at 34° C. for 2 hours, centrifuged at 5,000×g for 5 minutes, and the protoplasting solution discarded. The cell pellet was resuspended in a homogenization buffer consisting of 0.6 M sorbitol, 0.06 M Tris pH 7.5, 1 mM EDTA, and 0.1% BSA. Protoplasts were disrupted with a mechanical tissue homogenizer (Tekmar, Cincinnati, Ohio). After one centrifugation of the lysate at 2500×g for 10 minutes at 4° C., the pellet was discarded and the centrifugation was repeated. The supernatant from this second centrifugation was collected and re-centrifuged at 116,000×g for 1 hour. The final pellet was resuspended in the homogenization buffer before being subjected to further analysis.

Measurements of Membrane Fluidity

The relative fluidity of isolated microsomes was determined by steady-state fluorescence polarization measurements of membranes containing the hydrophobic fluoroprobe DPH (1,6-diphenyl -1,3,5-hexatriene), according to McCourt et al. (Plant Physiol., 84:353-357, 1987). The fatty acid content of the microsomal membrane preparations was determined by FAME analysis using a 17:0 methyl ester of known concentration as internal standard. DPH in solution in tetrahydrofurane was added to microsomes to achieve a molar ratio DPH/lipid of 1/500 and incubated for 45 minutes at 4° C. The suspension washed with 10 mM Tricine pH 7.9, 10 mM NaCl, 100 mM sorbitol, and centrifuged at 116,000×g for 40 minutes. The pellets were resuspended in Tricine buffer to a final concentration of 1 μM DPH and fluorescence polarization measurements were carried out on an SLM4800 spectrofluorometer (Spectronic Instruments, Rochester, N.Y.) at several temperatures between 10° C. and 40° C. Excitation was provided by light at 360 nm with a band pass of 2 nm. The emission was collected in the T-format without monochrometers using cut-off filters at 470 nm. Glan-Thompson (Santa Clara, Calif.) calcite polarizers were used. The data were analyzed using the software supplied by SLM (Toronto, Canada). Membrane fluidity was expressed by calculating P=(r/r₀)^{1/2}, where r₀ is the theoretical limiting anisotropy in the absence of rotational motion, and r is the steady-state anisotropy measured in the membrane. In a fully ordered membrane, P=1, and the smaller the P value, the more fluid the membrane.

Stress Experiments

Transgenic yeast and control yeast transformed with the empty vector were grown aerobically at 25° C. in a complete minimal (CM) medium lacking uracil and in the presence of 2% glucose. Cell growth was followed by turbidity measurements at 600 nm. Cells were harvested during the exponential phase when the optical density was between 0.1 and 1.0, corresponding to a cell density of 3×10⁶ to 3×10⁷ cfu/mL. Cells were washed twice in 67 mM phosphate buffer and resuspended in the original volume prior to exposure to stress conditions. Cells were treated in 10% ethanol (v/v) or 3 mM hydrogen peroxide for 8 hours. Cell viability was determined by appropriate dilution followed by plating of triplicate samples on CM agar. Colonies were counted after 2 days incubation at 28° C. Stress tolerance, expressed as percentage survivors, was determined by comparing the colony count of stressed cells to that of unstressed controls.

B. Results

Cloning and Characterization of a New Fatty Acid Desaturase Gene

A database search using Arabidopsis FAD2, FAD6, and FAD7 desaturases as queries, revealed a number of high-scoring Expressed Sequence Tags (ESTs) from C. elegans. Some of these were identical to the previously described fat-1, which encodes an ω-3 desaturase (Spychalla et al., Proc. Natl. Acad. Sci. USA, 94:1142-1147, 1997). However, several with high scores differed significantly from fat-1, and alignment of these sequences indicated that they originated from a single gene. Of these sequences, NCBI-57754 (D34903), NCBI-6233 (M89244), NCBI-55444 (D32410), NCBI-6197 (M89208) and NCBI-5424 (Z 14917), the clone with the most sequence information was NCBI-6197 (CEL18F3). This clone was obtained from the C. elegans Genome Sequencing Center. The insert from this clone was radiolabeled and used to probe approximately 50,000 plaques of a C. elegans, mixed-stage cDNA library. The screen yielded 20 positive clones with the longest cDNA insert being 1.3 kb in length as judged by agarose gel electrophoresis. One of these long clones, pCM18, was completely sequenced and found to contain a 1284 bp cDNA insert. The cDNA encoded an open reading frame for a protein predicted to consist of 376 residues, with a molecular mass of 43.3 kDa. Alignment of the predicted protein with known desaturase proteins revealed a sequence identity of 51% with FAT-1, 32% with FAD2, and 31% with FAD3; 56 amino acids were conserved in all four sequences (FIG. 1). Based on this homology to known desaturases, the protein was designated FAT-2 (fatty acid desaturase-2). Among the conserved residues were the 8 histidines that occur in most membrane desaturases, and have been shown to be important for desaturase activity (Shanklin et al., Biochemistry, 33:12787-12794, 1994). The arrangement of these residues in three histidine-rich sequence motifs with conserved spacing between the motifs is characteristic of the membrane-bound desaturases. The first motif, HXXXH, starts at residue 93 of the FAT-2 sequence, the second HXXHH at residue 129, and the third HXXHH at residue 295. The FAT-2 protein also contains the sequence KAKKAQ at its carboxyl terminus, which is similar to the proposed endoplasmic reticulum (ER) retention signal KXKXX common to many transmembrane ER proteins (Jackson et al., Embo J., 9:3153-3162, 1990). This sequence analysis indicated that the pCM18 cDNA encoded a fatty acid desaturase or an enzyme with a closely related function. Since the predicted FAT-2 protein is equally similar both to the Arabidopsis FAD2 Δ¹²-desaturase and to the FAD3 ω-3 desaturase (FIG. 1), the function of FAT-2 could not be deduced from sequence analysis alone. Because the previously characterized FAT-1 is an ω-3 desaturase, it seemed likely that FAT-2 represented the C. elegans Δ¹²-desaturase.

Functional Expression of FAT-2 in Yeast

The inventors have expressed the fat-2 cDNA in S. cerevisiae, which normally produces only mono-unsaturated 16:1Δ⁹ and 18:1Δ⁹ fatty acids. Expression of the fat-2 cDNA in yeast allows examination of the activity of the FAT-2 protein, since S. cerevisiae contains substantial amounts of both 16:1Δ⁹ and 18:1Δ⁹ fatty acids in its membrane lipids.

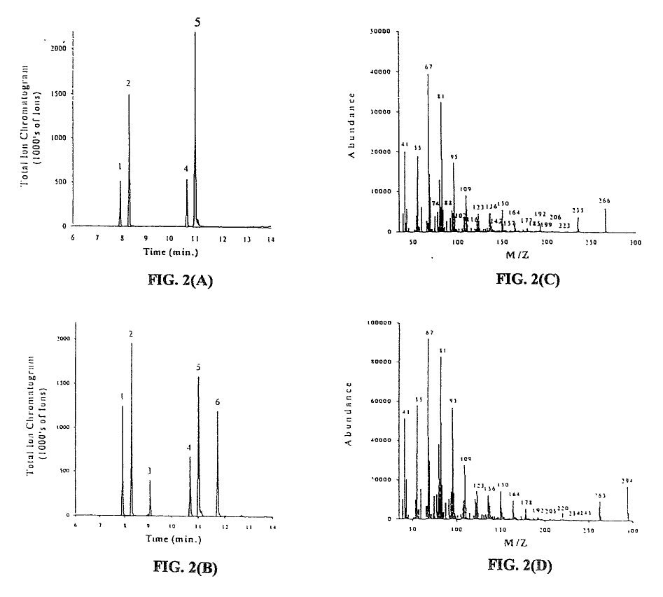

The plasmid pMK195-fat-2, expressing the cDNA under control of the ADH1 promoter, was transformed into yeast cells by selection for uracil prototrophy and grown on uracil-deficient medium. As a control, the empty pMK195 vector was transformed and cultured in parallel. After two days of culture at 28° C. the cells were harvested and FAMEs prepared. Analysis of the total fatty acids from the pMK195-fat-2-bearing strain revealed two peaks not present in the empty vector control strain. These peaks, with retention times of 8.48 and 11.096 minutes, represented apparent desaturation products from the common yeast fatty acids 16:1Δ⁹ and 18:1Δ⁹ (FIGS. 2(A), 2(B)). These desaturation products were identified as 16:2Δ^{9,12} and 18:2Δ^{9,12} by comparison of their mass spectra with those of commercial standards (FIGS. 2(C) and 2(D)). The molecular ion is correct for each fatty acid: 266 for 16:2Δ^{9,12}, and 294 for 18:2Δ^{9,12} (FIGS. 2(C), 2(D)). These polyunsaturated fatty acids accounted for 22% of the total fatty acids of yeast cells harvested during exponential growth, and increased to 46% when cultures entered stationary phase. Lipid analysis by thin layer chromatography indicated that polyunsaturated fatty acids accumulated in all of the major membrane phospholipids including phosphatidylcholine, phosphatidylethanolamine, and phosphatidylserine.

In summary, FAT-2 expressed in transgenic yeast recognizes both 16- and 18-carbon Δ⁹ substrates and converts up to 40% of these substrates to 16:2Δ^{9,12} and 18:2Δ^{9,12} (FIGS. 2(A)-2(B)).

Membrane Fluidity in Yeast Membranes Containing 16:2 and 18:2 Fatty Acids

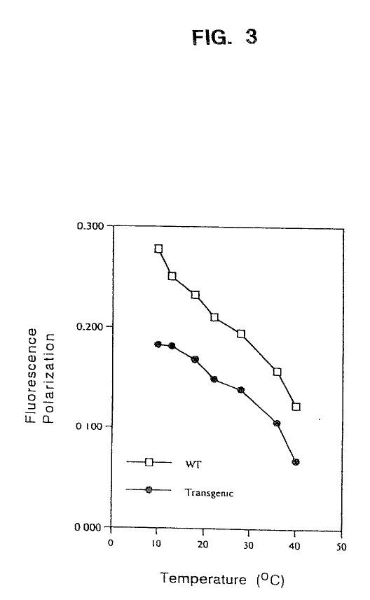

To determine if increased levels of desaturation in yeast membranes affected fluidity, we measured membrane fluidity by fluorescence polarization, using diphenylhexatriene (DPH) as a probe as described herein. After the measured fluorescence intensities were corrected for background fluorescence and light scattering from an unlabelled sample, the fluorescence polarization (P) was determined in membranes prepared from yeast transformed with pMK195-fat-2 and from the empty vector control strain. Polyunsaturated fatty acids in transgenic yeast microsomes used for the experiment accounted for 22% of total fatty acids. Throughout the entire temperature range used in the experiments, microsomes from cells expressing the FAT-2 desaturase showed substantially lower P values. These lower P values increased rotational mobility of the DPH probe and indicate an increase in the fluidity of the membrane bilayer at every temperature. The highest P values and greatest differential between control and FAT-2 membranes was observed at 10° C., the coldest temperature tested (FIG. 3).

Transgenic yeast expressing C. elegans fat-2, and thereby containing 16:2 and 18:2 fatty acids, exhibited a significantly more fluid membrane at all temperatures tested (FIG. 3).

Many organisms, including microorganisms and plants, alter the composition of their membrane lipids to compensate for the decrease of fluidity of the lipid bilayer at low temperatures (Russel, Trends Biochem. Sci., 9:108-112, 1984; Harwood et al., in Temperature Adaptation of Biological Membranes (Cossins, ed.), Portland Press, London, pp. 107-118, 1994). The homeoviscous adaptation of biological membranes is an environmentally triggered acclimation that is thought to improve membrane functionality at low temperature (McElhaney, Biomembranes, 12:249-276, 1984). However, the exact contribution of membrane unsaturation to low-temperature adaptation is not well understood (Cossins, in Temperature Adaptation of Biological Membranes (Cossins, ed.), Portland Press, London, pp. 63-76, 1994). The Arabidopsis thaliana fad2 mutant, which lacks the Δ¹²-desaturase activity, is unable to survive at low temperatures (Miquel et al., Proc. Natl. Acad. Sci. USA, 90:6208-6212, 1993). Likewise, the Fad12 mutant of the cyanobacterium Synechocystis PCC6803, which is deficient in Δ¹²-desaturase, grows more slowly than wild type at 22° C. although growth at 34° C. is unaffected (Wada and Murata, Plant Cell Physiol., 30:971-978, 1989). Thus, in both prokaryotic and eukaryotic organisms that contain high levels of polyunsaturated fatty acids, reductions in Δ¹²-desaturation and membrane polyunsaturation compromise cell function specifically at low temperatures. When the gene encoding Δ¹²-desaturase from Synechocystis PCC6803 (desA) was expressed in a cyanobacterium that normally contains only monounsaturated fatty acids (Synechococcus PCC7942), the membrane lipids of this organism became enriched with up to 25% polyunsaturated fatty acids (Wada et al., Nature, 347:200-203, 1990; and Wada et al., Proc. Natl. Acad. Sci. USA, 91:4273-4277, 1994). This large increase in membrane unsaturation was shown to reduce low-temperature damage to the photosynthetic machinery. However, this effect was small and no improvement in the growth rate of transformed cells was reported at any temperature (Wada et al., Nature, 347:200-203, 1990; and Wada et al., Proc. Natl. Acad. Sci. USA, 91:4273-4277, 1994).

Increased Stress Tolerance

There is a considerable body of literature correlating tolerance to cold, ethanol, and oxidative stress with membrane fluidity in a variety of yeast strains (Swan and Watson, Can. J. Microbiol., 43:70-77, 1997; Guerzoni et al., Can. J. Microbiol., 43:569-476, 1997; and Steels et al., Microbiology, 140:569-576, 1994); it is often argued that increased membrane fluidity should increase resistance to all of these stress factors (Steels et al., Microbiology, 140:569-576, 1994).

Temperature

The growth rate of both the experimental and control strains was examined over a range of temperatures to determine if membrane desaturation affected cold tolerance. Growth rates and fatty acid content of transformed yeast cells either expressing the fat-2 cDNA or containing the empty vector were measured at several temperatures between 4° C. and 30° C. At all temperatures between 15° C. and 30° C., yeast cells expressing FAT-2 had growth rates identical to the control strain. However, at 12° C., the growth rate of the FAT-2 expressing yeast was substantially higher than that of the control strain (0.022/hour vs. 0.014/hour, Table 1). At 4° C., growth of both strains was too slow to measure reliably.

The increased polyunsaturation of membranes in the fat-2 transgenic yeast confers a growth rate advantage to cells growing at 12° C., while no change is seen at higher temperatures (Table 1). For both the prokaryote Synechococcus PCC7942, and the eukaryote S. cerevisiae, the beneficial effects of providing polyunsaturated membranes are modest and confined to the lowest temperatures within the physiological temperature range for these organisms. Taken together, these observations indicate that membrane polyunsaturation may be essential for survival or optimum growth at low temperatures, but that polyunsaturation is only one feature required.

Ethanol

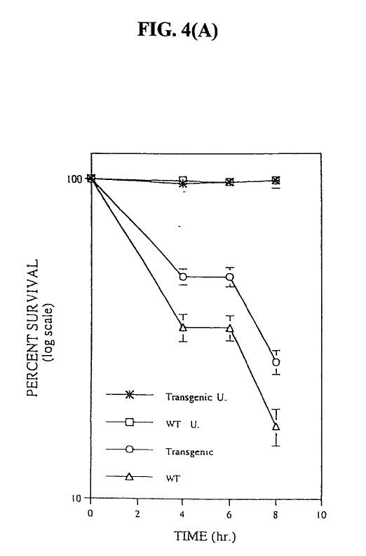

As a measure of resistance to ethanol stress, we measured viability under exposure to 10% ethanol. The yeast expressing FAT-2 exhibited viability twice that of control cells when exposed to 10% ethanol for 8 hours (FIG. 4(A)).

The ability to produce polyunsaturated fatty acids offered a more significant advantage to yeast cells subjected to ethanol stress. The viability of transgenic yeast expressing FAT-2 was twice that of control cells when exposed to 10% ethanol (FIG. 4(A)). Although S. cerevisiae is considered to be an ethanol-tolerant species, ethanol does inhibit cell growth, viability, solute accumulation, and proton fluxes at concentrations above the threshold of tolerance (Alexandre et al., FEMS Microbiol. Lett., 124:17-22, 1994). Ethanol stress is known to produce changes in the composition of the yeast plasma membrane including the levels and chain length of unsaturated fatty acids resulting in modification of membrane fluidity, and it has been suggested that these changes are specific responses that ameliorate the effect of ethanol (Gille et al., J. Gen. Microbiol., 139:1627-1634, 1993). However, attempts to test the possible correlation between membrane fatty acid composition or fluidity and ethanol tolerance have produced contradictory results (Swan and Watson, Can. J. Microbiol., 43:70-77, 1997; Guerzoni et al., Can. J. Microbiol., 43:569-476, 1997; and Swan and Watson, Can. J. Microbiol., 45:472-479, 1999). These studies are complicated by the fact that comparisons were made across different yeast strains or species, which can be expected to differ in many characteristics.

The results reported here were obtained by comparing control and transgenic cells that are isogenic except for the fat-2 cDNA. They show a distinct increase in viability for the transgenic cells containing polyunsaturated fatty acids and, in this respect, are consistent with previous studies in which yeast cells were grown in the presence of 18:2 (Thomas et al., Arch. Microbiol., 117:239-245, 1978), or were expressing a plant Δ¹²-desaturase (Kajiwara et al., Appl. Environ. Microbiol., 62:4309-4313, 1996).

Oxidation

To investigate the contribution of PUFAs to oxidative stress tolerance, we compared the ability of FAT-2 transformants and wild-type yeast cells to survive following hydrogen peroxide exposure. Yeast expressing FAT-2 survived 8 hours of treatment in 3 mM hydrogen peroxide at a rate more than twice as high as those of control cells under the same conditions (FIG. 4(B)). These results are consistent with previous suggestions that the presence of polyunsaturated fatty acids promotes increased tolerance to ethanol and oxidative stresses (Guerzoni et al., Can. J. Microbiol., 43:569-476, 1997; and Steels et al., Microbiology, 140:569-576, 1994).

The ability to produce polyunsaturated fatty acids also offered a significant advantage to yeast cells subjected to oxidative stress. The viability of transgenic yeast expressing FAT-2 was twice that of control cells when exposed to 3 mM hydrogen peroxide.

This increased tolerance to oxidative stress of yeast expressing FAT-2 (FIG. 4(B)) might involve fluidity changes within the plasma membrane or endomembranes of the cell. However, in general, tolerance to oxidative stress is known to involve enzyme-based detoxification and free-radical scavenging mechanisms that have been described from many different organisms (Gille et al., J. Gen. Microbiol., 139:1627-1634, 1993; Miller and Britigan, Clin. Microbiol. Rev., 10:1-18, 1997; Dixon et al., Curr. Op. Plant Biol., 1:258-266; 1998; Hogg, Semin. Reprod. Endocrinol., 16:241-248, 1998; and Reiter, FASEB J., 9:526-533, 1995). Typically, these mechanisms are strongly induced by mild oxidative stress. Because polyunsaturated fatty acids are considerably more susceptible to aerobic peroxidation and free-radical formation than monounsaturated or saturated fatty acids, it is likely that yeast cells expressing FAT-2 experience a mild, constitutive level of oxidative stress under normal culture conditions. It is possible, therefore, that polyunsaturated lipids provide increased protection against oxidative stress through the induction of endogenous tolerance mechanisms.

The following Table 1 lists fatty acid profiles and growth rates of yeast cells at different temperatures. Cells were transformed either with the control vector (C) or expressing FAT-2, grown on complete minimal medium lacking uracil until late log phase. They were harvested and fatty acid analysis of FAMEs was carried out by gas chromatography. Growth at 4° C. was too slow for accurate measurement. Numbers in the table are the weight-percent of the indicated fatty acids, as a fraction of total fatty acids.

TABLE 1

| Temperature | | | | |
|---|---|---|---|---|
| 4° C. | 12° C. | 15° C. | 22° C. | 30° C. |

| | Fatty Acids | | | | | | | | | |
|---|---|---|---|---|---|---|---|---|---|---|
| | C | FAT-2 | C | FAT-2 | C | FAT-2 | C | FAT-2 | C | Fat-2 |
| 16:0 + 18:0 | 17 | 19 | 19 | 20 | 19 | 20 | 18 | 20 | 25 | 27 |
| 16:1 + 18:1 | 81 | 77 | 80 | 61 | 80 | 62 | 79 | 44 | 71 | 41 |
| 16:2 + 18:2 | 0 | 5 | 0 | 17 | 0 | 16 | 0 | 32 | 0 | 26 |
| Growth rate | — | — | 0.014 | 0.022 | 0.031 | 0.033 | 0.10 | 0.10 | 0.11 | 0.11 |
| (h⁻¹) | | | | | | | | | | |

### III. EXAMPLES

### Example 1

Δ¹²-Desaturase Protein and Nucleic Acid Sequences

As described above, the invention provides desaturases and desaturase-specific nucleic acid sequences. With the provision herein of these desaturase sequences, the polymerase chain reaction (PCR) may now be utilized as a preferred method for identifying and producing nucleic acid sequences encoding the desaturases. For example, PCR amplification of the desaturase sequences may be accomplished either by direct PCR from a plant cDNA library or by Reverse-Transcription PCR (RT-PCR) using RNA extracted from plant cells as a template. Desaturase sequences may be amplified from plant genomic libraries, or plant genomic DNA. Methods and conditions for both direct PCR and RT-PCR are known in the art and are described in Innis et al., PCR Protocols: A Guide to Methods and Applications, Academic Press: San Diego, 1990.

The selection of PCR primers is made according to the portions of the cDNA (or gene) that are to be amplified. Primers may be chosen to amplify small segments of the cDNA, the open reading frame, the entire cDNA molecule or the entire gene sequence. Variations in amplification conditions may be required to accommodate primers of differing lengths; such considerations are well known in the art and are discussed in Innis et al., PCR Protocols: A Guide to Methods and Applications, Academic Press: San Diego, 1990; Sambrook et al. (eds.), Molecular Cloning: A Laboratory Manual 2nd ed., vol. 1-3, Cold Spring Harbor Laboratory Press, Cold Spring Harbor, N.Y., 1989; and Ausubel et al. (eds.) Current Protocols in Molecular Biology, John Wiley & Sons, New York (with periodic updates), 1998. By way of example, the cDNA molecules corresponding to additional desaturases may be amplified using primers directed towards regions of homology between the 5′ and 3′ ends of the prototypical C. elegans fat-2 sequence. Example primers for such a reaction are:

| | | |
|---|---|---|
| primer 1: | 5+ ATG ACA ATC GCT ACA 3+ | (SEQ ID NO: 3) |
| primer 2: | 5+ TTA TTG AGC CTT CTT 3+ | (SEQ ID NO: 4) |

These primers are illustrative only; one skilled in the art will appreciate that many different primers may be derived from the provided nucleic acid sequences. Re-sequencing of PCR products obtained by these amplification procedures is recommended to facilitate confirmation of the amplified sequence and to provide information on natural variation between desaturase sequences. Oligonucleotides derived from the desaturase sequence may be used in such sequencing methods.

Oligonucleotides that are derived from the desaturase sequences are encompassed within the scope of the present invention. Preferably, such oligonucleotide primers comprise a sequence of at least 10-20 consecutive nucleotides of the desaturase sequences. To enhance amplification specificity, oligonucleotide primers comprising at least 15, 20, 25, 30, 35, 40, 45, or 50 consecutive nucleotides of these sequences may also be used.

A. Desaturases in Other Animal Species

Orthologs of the FAT-2 gene are present in a number of other animals that are able to produce Δ¹² unsaturated fatty acids. With the provision herein of the FAT-2 nucleic acid sequences, the cloning by standard methods of cDNAs and genes that encode Δ¹²-desaturase orthologs in these other species is now enabled. As described above, orthologs of the disclosed Δ¹²-desaturase genes have Δ¹²-desaturase biological activity and are typically characterized by possession of at least 60% sequence identity counted over the full length alignment with the amino acid sequence of the disclosed Δ¹²-desaturase sequences using the NCBI Blast 2.0 (gapped blastp set to default parameters). Proteins with even greater similarity to the reference sequences will show increasing percentage identities when assessed by this method, such as at least 65%, at least 70%, at least 75%, at least 80%, at least 90%, or at least 95% sequence identity.

Both conventional hybridization and PCR amplification procedures may be utilized to clone sequences encoding desaturase orthologs. Common to both of these techniques is the hybridization of probes or primers that are derived from the Δ¹²-desaturase nucleic acid sequences. Furthermore, the hybridization may occur in the context of Northern blots, Southern blots, or PCR.

Direct PCR amplification may be performed on cDNA or genomic libraries prepared from any of various plant species, or RT-PCR may be performed using mRNA extracted from plant cells using standard methods. PCR primers will comprise at least 10 consecutive nucleotides of the Δ¹²-desaturase sequences. One of skill in the art will appreciate that sequence differences between the Δ¹²-desaturase nucleic acid sequence and the target nucleic acid to be amplified may result in lower amplification efficiencies. To compensate for this longer PCR primers or lower annealing temperatures may be used during the amplification cycle. Where lower annealing temperatures are used, sequential rounds of amplification using nested primer pairs may be necessary to enhance specificity.

For conventional hybridization techniques the hybridization probe is preferably conjugated with a detectable label such as a radioactive label, and the probe is preferably at least 10 nucleotides in length. As is well known in the art, increasing the length of hybridization probes tends to give enhanced specificity. The labeled probe derived from the Δ¹²-desaturase nucleic acid sequence may be hybridized to a plant cDNA or genomic library and the hybridization signal detected using methods known in the art. The hybridizing colony or plaque (depending on the type of library used) is then purified and the cloned sequence contained in that colony or plaque is isolated and characterized.

Orthologs of the C. elegans Δ¹²-desaturase alternatively may be obtained by immunoscreening of an expression library. With the provision herein of the disclosed C. elegans Δ¹²-desaturase nucleic acid sequences, the enzymes may be expressed and purified in a heterologous expression system (e.g., E. coli) and used to raise antibodies (monoclonal or polyclonal) specific for Δ¹²-desaturases. Antibodies may also be raised against synthetic peptides derived from the desaturase amino acid sequence presented herein. Methods of raising antibodies are well known in the art and are described generally in Harlow and Lane, Antibodies, A Laboratory Manual, Cold Springs Harbor Laboratory, 1988. Such antibodies can then be used to screen an expression cDNA library produced from a plant. This screening will identify the desaturase ortholog. The selected cDNAs can be confirmed by sequencing and enzyme activity assays.

B. Δ¹²-Desaturase Variants

With the provision of the C. elegans desaturase amino acid sequences (SEQ ID NO: 2) and the corresponding cDNA (SEQ ID NO: 1), variants of these sequences now can be created.

Variant desaturases include proteins that differ in amino acid sequence from the desaturase sequences disclosed (by one or more amino acids), but that retain desaturase biological activity. Such proteins may be produced by manipulating the nucleotide sequence encoding the desaturase using standard procedures such as site-directed mutagenesis or the polymerase chain reaction. The simplest modifications involve the substitution of one or more amino acids for amino acids having similar biochemical properties. These so-called “conservative substitutions” are likely to have minimal impact on the activity of the resultant protein. Table 2 shows amino acids that may be substituted for an original amino acid in a protein and that are regarded as conservative substitutions.

TABLE 2

| Original | Conservative |
|---|---|
| Residue | Substitutions |
| ala | Ser |
| arg | Lys |
| asn | gln; his |
| asp | Glu |
| cys | Ser |
| gln | Asn |
| glu | Asp |
| gly | Pro |
| his | asn; gln |
| ile | leu; val |
| leu | ile; val |
| lys | arg; gln; glu |
| met | leu; ile |
| phe | met; leu; tyr |
| ser | Thr |
| thr | Ser |
| trp | Tyr |
| tyr | trp; phe |
| val | ile; leu |

More substantial changes in enzymatic function or other features may be obtained by selecting substitutions that are less conservative than those in Table 2, i.e., selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions that, in general, are expected to produce the greatest changes in protein properties will be those in which: (a) a hydrophilic residue, e.g., seryl or threonyl, is substituted for (or by) a hydrophobic residue, e.g., leucyl, isoleucyl, phenylalanyl, valyl, or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine. The effects of these amino acid substitutions or deletions or additions may be assessed for desaturase derivatives by analyzing the ability of the derivative proteins to catalyze the desaturation of, for instance, 16:1Δ⁹ to 16:2Δ^{9,12}.

Variant desaturase cDNA or genes may be produced by standard DNA mutagenesis techniques, for example, M13 primer mutagenesis. Details of these techniques are provided in Sambrook et al. (eds.), Molecular Cloning: A Laboratory Manual, 2nd ed., vol. 1-3, Cold Spring Harbor Laboratory Press, Cold Spring Harbor, N.Y., 1989, Ch. 15. By the use of such techniques, variants may be created that differ in minor ways from the desaturase cDNA or gene sequences, yet that still encode a protein having desaturase biological activity. DNA molecules and nucleotide sequences that are derivatives of those specifically disclosed herein and that differ from those disclosed by the deletion, addition, or substitution of nucleotides while still encoding a protein having desaturase biological activity are comprehended by this invention. In their simplest form, such variants may differ from the disclosed sequences by alteration of the coding region to fit the codon usage bias of the particular organism into which the molecule is to be introduced.

Alternatively, the coding region may be altered by taking advantage of the degeneracy of the genetic code to alter the coding sequence in such a way that, even though the nucleotide sequence is substantially altered, it nevertheless encodes a protein having an amino acid sequence identical or substantially similar to the disclosed desaturase amino acid sequences. For example, the fourth amino acid residue of the FAT-2 cDNA (SEQ ID NO: 1) is alanine. This is encoded in the open reading frame (ORF) by the nucleotide codon triplet GCT. Because of the degeneracy of the genetic code, three other nucleotide codon triplets—GCA, GCC, and GCG—also code for alanine. Thus, the nucleotide sequence of the ORF can be changed at this position to any of these three codons without affecting the amino acid composition of the encoded protein or the characteristics of the protein. Based upon the degeneracy of the genetic code, variant DNA molecules may be derived from the cDNA and gene sequences disclosed herein using standard DNA mutagenesis techniques as described above, or by synthesis of DNA sequences. Thus, this invention also encompasses nucleic acid sequences that encode the desaturase protein but that vary from the disclosed nucleic acid sequences by virtue of the degeneracy of the genetic code.

Variants of the desaturase also may be defined in terms of their sequence identity with the desaturase amino acid and nucleic acid sequences described supra. As described above, Δ¹²-desaturases have Δ¹²-desaturase biological activity and share at least 60% sequence identity with the disclosed Δ¹²-desaturase sequences. Nucleic acid sequences that encode such proteins may be determined readily by applying the genetic code to the amino acid sequence of the desaturase, and such nucleic acid molecules may be produced readily by assembling oligonucleotides corresponding to portions of the sequence.

As previously mentioned, another method of identifying variants of the desaturase is nucleic acid hybridization. Nucleic acid molecules that are derived from the desaturase cDNA and gene sequences include molecules that hybridize under various conditions to the disclosed C. elegans Δ²-desaturase nucleic acid molecules, or fragments thereof. Generally, hybridization conditions are classified into categories, for example very high stringency, high stringency, and low stringency. The conditions for probes that are about 600 base pairs or more in length are provided above. The sequences encoding the desaturase identified through hybridization may be incorporated into transformation vectors and introduced into host cells to produce the respective desaturase.

### Example 2

Production of Recombinant Δ¹²-Desaturase in Heterologous Expression Systems

Various yeast strains and yeast-derived vectors are commonly used for the expression of heterologous proteins. For instance, Pichia pastoris expression systems, obtained from Invitrogen (Carlsbad, Calif.), may be used to practice the present invention. Such systems include suitable P. pastoris strains, vectors, reagents, transformants, sequencing primers, and media. Available strains include KM71H (a prototrophic strain), SMD1168H (a prototrophic strain), and SMD1168 (a pep4 mutant strain) (Invitrogen Product Catalogue, 1998, Invitrogen, Carlsbad Calif.).

Non-yeast eukaryotic vectors may be used with equal facility for expression of proteins encoded by modified nucleotides according to the invention. Mammalian vector/host cell systems containing genetic and cellular control elements capable of carrying out transcription, translation, and post-translational modification are well known in the art. Examples of such systems are the well-known baculovirus system, the ecdysone-inducible expression system that uses regulatory elements from Drosophila melanogaster to allow control of gene expression, and the sindbis viral-expression system that allows high-level expression in a variety of mammalian cell lines, all of which are available from Invitrogen, Carlsbad, Calif.

The cloned expression vector encoding at least one Δ¹²-desaturase may be transformed into any of various cell types for expression of the cloned nucleotide. Many different types of cells may be used to express modified nucleic acid molecules. Examples include cells of yeasts, fungi, insects, mammals, and plants, including transformed and non-transformed cells. For instance, common mammalian cells that could be used include HeLa cells, SW-527 cells (ATCC deposit #7940), WISH cells (ATCC deposit #CCL-25), Daudi cells (ATCC deposit #CCL-213), Mandin-Darby bovine kidney cells (ATCC deposit #CCL-22) and Chinese hamster ovary (CHO) cells (ATCC deposit #CRL-2092). Common yeast cells include Pichia pastoris (ATCC deposit #201178) and Saccharomyces cerevisiae (ATCC deposit #46024). Insect cells include cells from Drosophila melanogaster (ATCC deposit #CRL-10191), the cotton bollworm (ATCC deposit #CRL-9281), and Trichoplusia ni egg cell homoflagellates. Fish cells that may be used include those from rainbow trout (ATCC deposit #CLL-55), salmon (ATCC deposit #CRL-1681), and zebrafish (ATCC deposit #CRL-2147). Amphibian cells that may be used include those of the bullfrog, Rana catesbelana (ATCC deposit #CLL-41). Reptile cells that may be used include those from Russell's viper (ATCC deposit #CCL-140). Plant cells that could be used include Chlamydomonas cells (ATCC deposit #30485), Arabidopsis cells (ATCC deposit #54069) and tomato plant cells (ATCC deposit #54003). Many of these cell types are commonly used and are available from the ATCC as well as from commercial suppliers such as Pharmacia (Uppsala, Sweden), and Invitrogen (Carlsbad, Calif.).

Expressed protein may be accumulated within a cell or may be secreted from the cell. Such expressed protein may then be collected and purified. This protein may then be characterized for activity and stability and may be used to practice any of the various methods according to the invention.

### Example 3

Introduction of Δ¹²-Desaturase into Plants

Using the methods described herein, Δ¹²-desaturases of the invention can be cloned and expressed in plants to produce plants with enhanced amounts of polyunsaturated fatty acids. Such plants provide an inexpensive and convenient source of these important fatty acids in a readily harvestable and edible form.

For instance, the Δ¹²-desaturases of the invention could be cloned into a common food crop, such as corn, wheat, potato, tomato, yams, apples, pears, or into oil-seed plants such as sunflower, rapeseed, soy, or peanut plants. The resulting plant would express the appropriate enzyme that would catalyze the formation of polyunsaturated fatty acids. In the case of an oil-seed plant, the seed oil would be a rich source of Δ¹²-desaturated polyunsaturated fatty acids.

Standard techniques may be used to express an identified cDNA in transgenic plants in order to modify a particular plant characteristic. The basic approach is to clone the cDNA into a transformation vector such that the cDNA is operably linked to control sequences (e.g., a promoter) directing expression of the cDNA in plant cells. The transformation vector is then introduced into plant cells by any of various techniques (e.g., electroporation, particle bombardment, etc.) and progeny plants containing the introduced cDNA are selected. Preferably all or part of the transformation vector stably integrates into the genome of the plant cell. That part of the transformation vector that integrates into the plant cell and that contains the introduced cDNA and associated sequences for controlling expression (the introduced “transgene”) may be referred to as the recombinant expression cassette.

Selection of progeny plants containing the introduced transgene may be made based upon the detection of an altered phenotype. Such a phenotype may result directly from the cDNA cloned into the transformation vector or may be manifested as enhanced resistance to a chemical agent (such as an antibiotic) as a result of the inclusion of a dominant selectable marker gene incorporated into the transformation vector.

Successful examples of the modification of plant characteristics by transformation with cloned cDNA sequences are replete in the technical and scientific literature. Selected examples, which serve to illustrate the knowledge in this field of technology include:

U.S. Pat. No. 5,571,706 (“Plant Virus Resistance Gene and Methods”)

U.S. Pat. No. 5,677,175 (“Plant Pathogen Induced Proteins”)

U.S. Pat. No. 5,510,471 (“Chimeric Gene for the Transformation of Plants”)

U.S. Pat. No. 5,750,386 (“Pathogen-Resistant Transgenic Plants”)

U.S. Pat. No. 5,597,945 (“Plants Genetically Enhanced for Disease Resistance”)

U.S. Pat. No. 5,589,615 (“Process for the Production of Transgenic Plants with Increased Nutritional Value Via the Expression of Modified 2S Storage Albumins”)

U.S. Pat. No. 5,750,871 (“Transformation and Foreign Gene Expression in Brassica Species”)

U.S. Pat. No. 5,268,526 (“Overexpression of Phytochrome in Transgenic Plants”)

U.S. Pat. No. 5,262,316 (“Genetically Transformed Pepper Plants and Methods for their Production”)

U.S. Pat. No. 5,569,831 (“Transgenic Tomato Plants with Altered Polygalacturonase Isoforms”)

These examples include descriptions of transformation vector selection, transformation techniques, and the construction of constructs designed to over-express the introduced cDNA. In light of the foregoing and the provision herein of the desaturase amino acid sequences and nucleic acid sequences, it is thus apparent that one of skill in the art will be able to introduce the cDNAs, or homologous or derivative forms of these molecules, into plants in order to produce plants having enhanced desaturase activity. Furthermore, the expression of one or more desaturases in plants may give rise to plants having altered and/or increased desaturated fatty acid production.

A. Vector Construction, Choice of Promoters

A number of recombinant vectors suitable for stable transfection of plant cells or for the establishment of transgenic plants have been described including those described in Weissbach and Weissbach, Methods for Plant Molecular Biology, Academic Press, 1989; and Gelvin et al., Plant and Molecular Biology Manual, Kluwer Academic Publishers, 1990. Typically, plant-transformation vectors include one or more cloned plant genes (or cDNAs) under the transcriptional control of 5′- and and 3′-regulatory sequences and a dominant selectable marker. Such plant transformation vectors typically also contain a promoter regulatory region (e.g., a regulatory region controlling inducible or constitutive, environmentally or developmentally regulated, or cell- or tissue-specific expression), a transcription initiation start site, a ribosome binding site, an RNA processing signal, a transcription termination site, and/or a polyadenylation signal.

Examples of constitutive plant promoters that may be useful for expressing the cDNA include: the cauliflower mosaic virus (CaMV) 35S promoter, which confers constitutive, high-level expression in most plant tissues (see, e.g., Odel et al., Nature, 313:810, 1985; Dekeyser et al., Plant Cell, 2:591, 1990; Terada and Shimamoto, Mol. Gen. Genet., 220:389, 1990; and Benfey and Chua, Science, 250:959-966, 1990); the nopaline synthase promoter (An et al., Plant Physiol., 88:547, 1988); and the octopine synthase promoter (Fromm et al., Plant Cell, 1:977, 1989).

Any of a variety of plant-gene promoters that are regulated in response to environmental, hormonal, chemical, and/or developmental signals also can be used for expression of the cDNA in plant cells, including promoters regulated by: (a) heat (Callis et al., Plant Physiol., 88:965, 1988; Ainley, et al., Plant Mol. Biol, 22:13-23, 1993; and Gilmartin et al., Plant Cell, 4:839-949, 1992); (b) light (e.g, the pea rbcS-3A promoter, Kuhlemeier et al., Plant Cell, 1:471, 1989, and the maize rbcS promoter, Schaffner and Sheen, Plant Cell, 3:997, 1991); (c) hormones, such as abscisic acid (Marcotte et al., Plant Cell, 1:969, 1989); (d) wounding (e.g., wunI, Siebertz et al., Plant Cell, 1:961, 1989); and (e) chemicals such as methyl jasmonate or salicylic acid (Gatz et al., Ann. Rev. Plant Physiol. Plant Mol. Biol., 48:9-108, 1997).

Alternatively, tissue-specific (root, leaf, flower, and seed, for example) promoters (Carpenter et al., Plant Cell, 4:557-571, 1992; Denis et al., Plant Physiol., 101:1295-1304, 1993; Opperman et al., Science, 263:221-223, 1993; Stockhause et al., Plant Cell, 9:479-489, 1997; Roshal et al., Embo. J., 6:1155, 1987; Schernthaner et al., Embo J., 7:1249, 1988; and Bustos et al., Plant Cell, 1:839, 1989) can be fused to the coding sequence to obtain a particular expression in respective organs. Where enhancement of production of desaturated fatty acid is desired in a seed (e.g., an oilseed) of a plant, the use of a seed-specific promoter is beneficial. For example, the napin promoter is an appropriate seed-storage protein promoter from Brassica that allows expression specific to developing seeds. The β-conglycinin promoters also can drive the expression of recombinant nucleic acids, thereby allowing the Δ¹²-desaturases of the invention to be expressed only in specific tissues, for example, seed tissues.

Alternatively, the native desaturase gene promoters may be utilized. With the provision herein of the desaturase nucleic acid sequences, one of skill in the art will appreciate that standard molecular biology techniques can be used to determine the corresponding promoter sequences. One of skill in the art will also appreciate that less than the entire promoter sequence may be used in order to obtain effective promoter activity. The determination of whether a particular region of this sequence confers effective promoter activity may readily be ascertained by operably linking the selected sequence region to a desaturase cDNA (in conjunction with suitable 3′-regulatory region, such as the NOS 3′-regulatory region as discussed below) and determining whether the desaturase is expressed.

Plant-transformation vectors may also include RNA-processing signals, for example, introns, that may be positioned upstream or downstream of the ORF sequence in the transgene. In addition, the expression vectors may also include additional regulatory sequences from the 3′-untranslated region of plant genes, e.g., a 3′-terminator region to increase mRNA stability of the mRNA, such as the PI-II terminator region of potato or the octopine or nopaline synthase (NOS) 3′-terminator regions. The native desaturase gene 3′-regulatory sequence may also be employed.

Finally, as noted above, plant-transformation vectors may also include dominant selectable marker genes to allow for the ready selection of transformants. Such genes include those encoding antibiotic-resistance genes (e.g., resistance to hygromycin, kanamycin, bleomycin, G418, streptomycin or spectinomycin) and herbicide-resistance genes (e.g., phosphinothricin acetyltransacylase).

B. Arrangement of Δ¹²-Desaturase Sequence in a Vector

The particular arrangement of the desaturase sequence in the transformation vector is selected according to the type of expression of the sequence that is desired.

Where enhanced desaturase activity is desired, the desaturase ORF may be operably linked to a constitutive high-level promoter such as the CaMV 35S promoter. As noted above, enhanced desaturase activity may also be achieved by introducing into a plant a transformation vector containing a variant form of the desaturase cDNA or gene, for example a form that varies from the exact nucleotide sequence of the desaturase ORF, but that encodes a protein retaining desaturase biological activity.

C. Transformation and Regeneration Techniques

Transformation and regeneration of both monocotyledonous and dicotyledonous plant cells are now routine, and the practitioner can determine the appropriate transformation technique. The choice of method varies with the type of plant to be transformed; those skilled in the art will recognize the suitability of particular methods for given plant types. Suitable methods may include, but are not limited to: electroporation of plant protoplasts; liposome-mediated transformation; polyethylene glycol (PEG)-mediated transformation; transformation using viruses; micro-injection of plant cells; micro-projectile bombardment of plant cells; vacuum infiltration; and Agrobacterium tumefaciens (AT)-mediated transformation. Typical procedures for transforming and regenerating plants are described in the patent documents listed at the beginning of this section.

By way of example only, transformation of Arabidopsis is achieved using, for example, Agrobacterium-mediated vacuum-infiltration process (Katavic et al., Mol. Gen. Genet., 245:363-370, 1994) or by the floral dip modification of it (Clough and Bent, Plant J., 16:735-743, 1998).

D. Selection of Transformed Plants

Following transformation and regeneration of plants with the transformation vector, transformed plants can be selected using a dominant selectable marker incorporated into the transformation vector. Typically, such a marker confers antibiotic resistance on the seedlings of transformed plants, and selection of transformants can be accomplished by exposing the seedlings to appropriate concentrations of the antibiotic.

After transformed plants are selected and grown to maturity, they can be assayed using the methods described herein to assess production levels of Δ¹²-desaturase protein and the level of Δ¹²-desaturase activity.

### Example 4

Creation of Δ¹²-Desaturase-Specific Binding Agents

Antibodies to the Δ¹²-desaturase enzymes, and fragments thereof, of the present invention may be useful for purification of the enzymes, as well as for other purposes. The provision of the desaturase sequences allows for the production of specific antibody-based binding agents to these enzymes.

Monoclonal or polyclonal antibodies may be produced to the desaturases, portions of the desaturases, or variants, orthologs or homologs thereof. Optimally, antibodies raised against epitopes on these antigens will specifically detect the enzyme. That is, antibodies raised against the C. elegans Δ¹²-desaturase would recognize and bind the C. elegans Δ¹²-desaturase, and would not substantially recognize or bind to other proteins. The determination that an antibody specifically binds to an antigen is made by any one of a number of standard immunoassay methods; for instance, Western blotting, Sambrook et al. (eds.), Molecular Cloning: A Laboratory Manual, 2nd ed., vol. 1-3, Cold Spring Harbor Laboratory Press, Cold Spring Harbor, N.Y., 1989.

To determine that a given antibody preparation (such as a preparation produced in a mouse against FAT-2) specifically detects the desaturase by Western blotting, total cellular protein is extracted from cells and electrophoresed on an SDS-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase; application of 5-bromo-4-chloro-3-indolyl phosphate/nitro blue tetrazolium results in the production of a densely blue-colored compound by immuno-localized alkaline phosphatase.

Antibodies that specifically detect a Δ²-desaturase will, by this technique, be shown to bind substantially only the desaturase band (having a position on the gel determined by the molecular weight of the desaturase). Non-specific binding of the antibody to other proteins may occur and may be detectable as a weaker signal on the Western blot (which can be quantified by automated radiography). The non-specific nature of this binding will be recognized by one skilled in the art by the weak signal obtained on the Western blot relative to the strong primary signal arising from the specific anti-desaturase binding.

Antibodies that specifically bind to desaturases belong to a class of molecules that are referred to herein as “specific binding agents.” Specific binding agents that are capable of specifically binding to the desaturase of the present invention may include polyclonal antibodies, monoclonal antibodies and fragments of monoclonal antibodies such as Fab, F(ab′)₂, and Fv fragments, as well as any other agent capable of specifically binding to one or more epitopes on the proteins.

Substantially pure Δ¹²-desaturase suitable for use as an immunogen can be isolated from transfected cells, transformed cells, or from wild-type cells. Concentration of protein in the final preparation is adjusted, for example, by concentration on an Amicon (Millipore, Bedford, Mass.) filter device, to the level of a few micrograms per milliliter. Alternatively, peptide fragments of a desaturase may be utilized as immunogens. Such fragments may be chemically synthesized using standard methods, or may be obtained by cleavage of the whole desaturase enzyme followed by purification of the desired peptide fragments. Peptides as short as three or four amino acids in length are immunogenic when presented to an immune system in the context of a major histocompatibility complex (MHC) molecule, such as MHC class I or MHC class II. Accordingly, peptides comprising at least 3 and preferably at least 4, 5, 6 or more consecutive amino acids of the disclosed desaturase amino acid sequences may be employed as immunogens for producing antibodies.

Because naturally occurring epitopes on proteins frequently comprise amino acid residues that are not adjacently arranged in the peptide when the peptide sequence is viewed as a linear molecule, it may be advantageous to utilize longer peptide fragments from the desaturase amino acid sequences for producing antibodies. Thus, for example, peptides that comprise at least 10, 15, 20, 25, or 30 consecutive amino acid residues of the amino acid sequence may be employed. Monoclonal or polyclonal antibodies to the intact desaturase, or peptide fragments thereof may be prepared as described below.

A. Monoclonal Antibody Production by Hybridoma Fusion

Monoclonal antibodies to any of various epitopes of the desaturase enzymes that are identified and isolated as described herein can be prepared from murine hybridomas according to the classic method of Kohler & Milstein (Nature, 256:495, 1975) or a derivative method thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol., 70:419, 1980) or a derivative method thereof. Selected positive clones can be expanded and their monoclonal antibody product harvested for use. Detailed procedures for monoclonal antibody production are described in Harlow & Lane (Antibodies, A Laboratory Manual, Cold Spring Harbor Laboratory, New York, 1988).

B. Polyclonal Antibody Production by Immunization

Polyclonal antiserum containing antibodies to heterogenous epitopes of a single protein can be prepared by immunizing suitable animals with the expressed protein, which can be unmodified or modified, to enhance immunogenicity. Effective polyclonal antibody production is affected by many factors related both to the antigen and the host species. For example, small molecules tend to be less immunogenic than other molecules and may require the use of carriers and an adjuvant. Also, host animals vary in response to site of inoculations and dose, with either inadequate or excessive doses of antigen resulting in low-titer antisera. Small doses (ng level) of antigen administered at multiple intradermal sites appear to be most reliable. An effective immunization protocol for rabbits can be found in Vaitukaitis et al., J. Clin. Endocrinol. Metab., 33:988-991, 1971.

Booster injections can be given at regular intervals, and antiserum harvested when the antibody titer thereof, as determined semi-quantitatively, for example, by double immunodiffusion in agar against known concentrations of the antigen, begins to fall. See, for example, Ouchterlony et al., Handbook of Experimental Immunology, Wier, D. (ed.), Chapter 19, Blackwell, 1973. A plateau concentration of antibody is usually in the range of 0.1 to 0.2 mg/mL of serum (about 12 μM). Affinity of the antisera for the antigen is determined by preparing competitive binding curves using conventional methods.

C. Antibodies Raised by Injection of cDNA

Antibodies may be raised against the desaturases of the present invention by subcutaneous injection of a DNA vector that expresses the enzymes in laboratory animals, such as mice. Delivery of the recombinant vector into the animals may be achieved using a hand-held form of the Biolistic system (Sanford et al., Particulate Sci. Technol., 5:27-37, 1987, as described by Tang et al., Nature (London), 356:153-154, 1992). Expression vectors suitable for this purpose may include those that express the cDNA of the enzyme under the transcriptional control of either the human β-actin promoter or the cytomegalovirus (CMV) promoter. Methods of administering naked DNA to animals in a manner resulting in expression of the DNA in the body of the animal are well known and are described, for example, in U.S. Pat. No. 5,620,896 (“DNA Vaccines Against Rotavirus Infections”); U.S. Pat. No. 5,643,578 (“Immunization by Inoculation of DNA Transcription Unit”); and U.S. Pat. No. 5,593,972 (“Genetic Immunization”), and references cited therein.

D. Antibody Fragments

Antibody fragments may be used in place of whole antibodies and may be readily expressed in prokaryotic host cells. Methods of making and using immunologically effective portions of monoclonal antibodies, also referred to as “antibody fragments,” are well known and include those described in Better & Horowitz, Methods Enzymol., 178:476-496, 1989; Glockshuber et al., Biochemistry, 29:1362-1367, 1990; and U.S. Pat. No. 5,648,237 (“Expression of Functional Antibody Fragments”); U.S. Pat. No. 4,946,778 (“Single Polypeptide Chain Binding Molecules”); and U.S. Pat. No. 5,455,030 (“Immunotherapy Using Single Chain Polypeptide Binding Molecules”), and references cited therein.

### Example 5

Δ¹²-Desaturase Production in vivo

The creation of recombinant vectors and transgenic organisms expressing the vectors are important for controlling the production of desaturases. These vectors can be used to decrease desaturase production, or to increase desaturase production. A decrease in desaturase production will likely result from the inclusion of an antisense sequence or a catalytic nucleic acid sequence that targets the desaturase encoding nucleic acid sequence. Conversely, increased production of desaturase can be achieved by including at least one additional desaturase encoding sequence in the vector. These vectors can then be introduced into a host cell, thereby altering desaturase production. In the case of increased production, the resulting desaturase may be used in in vitro systems, as well as in vivo for increased production of Δ¹²-desaturated fatty acids.

Increased production of Δ¹²-desaturated fatty acids in vivo can be accomplished by transforming a host cell, such as one derived from a plant, specifically an oilseed plant, with a vector containing at least one nucleic acid sequences encoding at least one Δ¹²-desaturase. Furthermore, the heterologous or homologous desaturase sequences can be placed under the control of a constitutive promoter, or an inducible promoter. This will lead to the increased production of Δ¹²-desaturase, thus altering production of desaturated fatty acids, especially altering the Δ¹²-desaturation in such molecules.

### Example 6

Expression of Fat2 Δ¹²-Fatty Acid Desaturase in Arabidopsis thaliana to Produce Increased Desaturation of Fatty Acids in Plant Seeds

Plant-Transformation Constructs

Plant-transformation vectors can be constructed, by standard DNA cloning techniques, to introduce the fat2 cDNA into plants so that the desaturase protein is expressed during seed development.

First, the Δ¹²-desaturase cDNA (SEQ ID NO: 1) can be engineered so as to be under the control of (functionally linked to) a plant promoter chosen because it is active during Arabidopsis seed development. For example, the promoter for phaseolin (van der Geest and Hall, Plant Mol. Biol., 32:579-588, 1996) or the promoter for napin (Stalberg et al., Plant Mol. Biol., 23:671-683, 1993) could be used. Promoters cloned specifically for this purpose also could be used. Appropriate promoters include those found on the genomic BAC clone T24A18 (LOCUS ATT24A18. 45980 bp Arabidopsis thaliana DNA chromosome 4, ESSA project, Accession No.: AL035680NID g4490701,1999) of the Arabidopsis genome, which regulate seed storage proteins of Arabidopsis, and promoters that express other genes specifically in seeds (Parcy et al., Plant Cell, 6:1567-1582, 1994).

The seed-specific promoter-desaturase construct(s) then can be transferred to one or more standard plant transformation T-DNA vectors, such as or similar to pART27 (Gleave, Plant Mol. Biol., 20:1203-1207, 1992), pGPTV (Becker et al., Plant Mol. Biol, 20:1195-1197, 1992), or pJIT119 (Guerineau et al., Plant Mol. Biol., 15:127-136, 1990).

Plant Transformation Procedures

Constructs produced as described can be used to transform Arabidopsis thaliana by the standard Agrobacterium-mediated vacuum-infiltration process (Katavic et al., Mol. Gen. Genet., 245:363-370, 1994) or by the floral dip modification of it (Clough and Bent, Plant J., 16:735-743, 1998). After the transformation process, seeds can be harvested from the plants when the plants mature. Transgenic progeny can be identified by selection using the appropriate antibiotic or herbicide. Plants that survive the transgenic selection can be grown to maturity and their seed harvested.

Analysis of Transgenic Plants

The seed of plants transformed by the construct containing the Δ¹²-desaturase can be analyzed by preparation of fatty acid methyl esters, followed by gas chromatography to determine their fatty acid composition. Plants expressing the Δ¹²-desaturase will desaturate the 18:1 (Δ⁹) fatty acid that occurs naturally in the Arabidopsis seed to 18:2 (Δ^{9,12}). At maturity, seed harvested from these transformed plants can be analyzed by gas chromatography. Seeds of plants expressing the Δ¹²-desaturase will contain increased levels of 18:2 fatty acid and decreased levels of 18:1 fatty acid.

By way of specific example, the following procedures were used to test the activity of fat-2 in plants. To express FAT-2 in Arabidopsis thaliana, the region encoding fat-2 was released from pCM18 (described above) by restriction digestion. The fragment containing the coding sequence was ligated into a corresponding restriction digest of the plasmid pART7 (Gleave, Plant Mol. Biol., 20:1203-1207, 1992.). This directional cloning procedure resulted in a construct, pART7-fat-2, which has the fat-2 coding sequence under control of the cauliflower mosaic virus 35S (CaMV) promoter, and upstream of the octopine synthetase plant terminator sequence.

This “plant-expression cassette,” consisting of plant promoter, coding sequence, and terminator, was transferred by restriction and ligation to the multiple cloning site of its companion vector, pART27 (Gleave, Plant Mol. Biol., 20:1203-1207, 1992), which is a T-DNA plant-transformation vector. Vector pART27 provides a selectable marker for plant transformation, the kanamycin resistance marker nptII, in its own plant-expression cassette. The multiple cloning site of the vector is between DNA sequences for the right and left T-DNA borders, so that genes cloned into the vector at the cloning sites can be transformed into plants. The construct, named pART27-fat-2, was confirmed by restriction analysis to have the correct structure and transformed by electroporation into the Agrobacterium tumefaciens strain GV3101 (Holsters et al., Plasmid 3:212-230, 1980) by selection for spectinomycin resistance, the bacterial selectable marker for pART27 derivatives.

Arabidopsis thaliana plants of the “columbia” ecotype were transformed with the resulting Agrobacterium strain using vacuum infiltration (Katavic et al., Mol. Gen. Genet., 245:363-370, 1994). After recovery of the plants, seed was harvested. Samples of the seed were sterilized and plated on standard plant medium, MS salts, supplemented with kanamycin at 50 μg ml⁻¹. After three weeks twelve of the surviving plants were transferred to soil, and named fat-2-L1, fat-2-L2, and so forth, through fat-2-L12. When these plants reached maturity, their seeds were harvested.

To screen for expression of the transgene, seeds from six of the individual plants were allowed to sprout on medium containing kanamycin. As a control, seeds from an established plant line (GUS control), which is kanamycin-resistant but wild-type with respect to its fatty acid composition, were sown on the identical medium. The fatty acid composition of root tissue from these plants was analyzed by derivitization of the fatty acids to fatty acid methyl esters (FAME) using 2.5% sulfuric acid in methanol, followed by gas chromatographic analysis using published techniques (Miquel and Browse, J. Biol. Chem., 267:1502-1509, 1992). The determinations were performed in duplicate. The analysis indicated that all six lines had increased levels of 18:2 in their vegetative root tissue as a result of FAT-2 expression (Table 3). The 18:2 fatty acids increased over a range of 1.4-fold to 1.6-fold in the six lines examined, establishing that expression of FAT-2 in Arabidopsis does increase the amount of polyunsaturated 18:2 fatty acid.

TABLE 3
Expression of fat-2 in transgenic
Arabidopsis increases the concentration
of 18:2 fatty acids in vegetative tissue.

| Trans- | | | | |
|---|---|---|---|---|
| genic | | fatty acid 18:2 as % of total fatty acids | | |
| Plant | Antibiotic | First | Second | Average |
| line | Resistance | determination | determination | determination |
| GUS | Kn | 20.1 | 17.4 | 18.8 |
| control | | | | |
| fat-2-L1 | Kn | 28.5 | 27.2 | 27.9 |
| fat-2-L2 | Kn | 27.7 | 26.9 | 27.3 |
| fat-2-L3 | Kn | 24.3 | 27.2 | 25.7 |
| fat-2-L5 | Kn | 28.0 | 33.4 | 30.7 |
| fat-2-L9 | Kn | 29.5 | 29.0 | 29.3 |
| fat-2-L10 | Kn | 30.6 | 27.4 | 29.0 |

Four of these lines were analyzed further, both to detect expression of the transgene in seeds and to determine if the inserted transgene was segregating in a Mendelian manner. Although the CaMV promoter is not very active in seeds, unique products of transgene expression may sometimes be detected (van de Loo et al., Proc. Natl. Acad. Sci. USA, 92:6743-6747, 1995). The fatty acid content of a single seed can be determined by FAME analysis. Accordingly, 12 seeds of each transgenic line and of wild-type Arabidopsis were individually analyzed, and the extent of transgene expression and its segregation pattern were characterized (Table 4). Since CaMV expression of transgenes is poor in seeds, it was not possible to accurately determine the degree of increase in the 18:2 content of the seeds. It is possible to detect the appearance of a new fatty acid in these seed from transformed plants, which is due to the expression of the fat-2 gene. This fatty acid, 20:2 (11,14), likely occurs when 18:2 produced by FAT-2 is elongated by the endogenous Arabidopsis seed metabolism. All four lines analyzed in this experiment had 20:2 fatty acid at low concentrations in some of their seed, while none was detectable in any wild type seed (Table 4). This establishes that the transgene, while not as strongly expressed as it would be with a seed-specific promoter, nonetheless alters seed fatty acid metabolism. Since the seeds in this generation represent a segregating Mendelian population, if the transgenic phenotype of 20:2 production is the result of insertion of the transgene at a single locus, then the expectation would be that three-quarters of the seed would have the phenotype. In fact, three of the four lines examined have approximately the three-quarters ratio predicted. The fourth had 20:2 in all seed tested, and may represent a multiple insertion event.

TABLE 4
Expression of fat-2 in transgenic Arabidopsis
produces the fatty acid 20:2
in seeds, and this effect can be used
to examine segregation of the
transgene.

| | | |
|---|---|---|
| fatty acid 20:2 | Number of seeds | Segregation |
| (% of total fatty acids | with 20:2 | ratio |

| | | | |
|---|---|---|---|
| WT | 0 | 0 of 12 | N/A |
| fat-2-L1 | 1.45 | 12 of 12 | 1.00 |
| fat-2-L2 | 0.99 | 8 of 10 | 0.80 |
| fat-2-L3 | 1.23 | 11 of 12 | 0.92 |
| fat-2-L5 | 1.78 | 8 of 12 | 0.75 |

Having illustrated and described the principles of the invention in multiple embodiments and examples, it should be apparent to those skilled in the art that the invention can be modified in arrangement and detail without departing from such principles. We claim all modifications coming within the spirit and scope of the following claims.

We Claim:

1. A purified desaturase protein, comprising an amino acid sequence selected from the group consisting of:
(a) an amino acid sequence as shown in SEQ. ID NO. 2;
(b) an amino acid sequence that differs from that specified in (a) by one or more conservative amino acid substitutions;
(c) an amino acid sequences having at least 60% sequence identity to the sequences specified in (a) or (b); and
(d) fragments of (a), (b), or (c),
wherein the purified protein has desaturase activity.

2. The desaturase protein of claim 1, wherein the desaturase protein is a Δ¹²-desaturase.

3. An isolated nucleic acid molecule, encoding a protein according to claim 1.

4. The isolated nucleic acid molecule of claim 3, comprising a sequence as shown in SEQ ID NO: 1.

5. A recombinant nucleic acid molecule, comprising a control sequence operably linked to the nucleic acid molecule of claim 3.

6. A cell, transformed with the recombinant nucleic acid molecule of claim 5.

7. The cell of claim 6, wherein the cell is a plant cell.

8. A transgenic organism, comprising a recombinant nucleic acid molecule according to claim 5, wherein the transgenic organism is selected from the group consisting of plants, bacteria, insects, fungi, and animals.

9. The transgenic organism of claim 8, wherein the organism is a plant.

10. The transgenic organism of claim 9, wherein the plant is selected from the group consisting of oil palm, sunflower, safflower, rapeseed, canola, soy, peanut, cotton, corn, rice, Arabidopsis, mustard, wheat, barley, potato, tomato, yam, apple, and pear plants.

11. An isolated nucleic acid molecule that:
(a) hybridizes under low-stringency conditions with a nucleic acid probe, the probe comprising a sequence as shown in SEQ ID NO: 1, and fragments thereof, and
(b) encodes a protein having desaturase activity.

12. A desaturase protein encoded by the nucleic acid molecule of claim 11.

13. The desaturase protein of claim 12, wherein the desaturase protein is a Δ¹²-desaturase.

14. A recombinant nucleic acid molecule, comprising a promoter sequence operably linked to the nucleic acid molecule of claim 11.

15. A cell transformed with the recombinant nucleic acid molecule of claim 14.

16. The cell of claim 15, wherein the cell is a plant cell.

17. A transgenic organism, comprising the transformed cell of claim 15, wherein the transgenic organism is selected from the group consisting of plants, bacteria, insects, fungi, and animals.

18. The transgenic organism of claim 17, that is a plant.

19. The transgenic organism of claim 18, wherein the plant is selected from the group consisting of oil palm, sunflower, safflower, rapeseed, canola, soy, peanut, cotton, corn, rice, Arabidopsis, mustard, wheat, barley, potato, tomato, yam, apple, and pear plants.

20. A specific binding agent that binds to the desaturase protein of claim 12.

21. An isolated nucleic acid molecule that:
(a) has at least 60% sequence identity with a nucleic acid sequence as shown in SEQ ID NO: 1; and
(b) encodes a protein having desaturase activity.

22. A method for identifying a nucleic acid sequence, comprising:
(a) hybridizing the nucleic acid sequence to at least 10 contiguous nucleotides of a sequence as shown in SEQ ID NO: 1; and
(b) identifying the nucleic acid sequence as corresponding to a nucleic acid encoding a desaturase.

23. The method of claim 22, wherein hybridizing the nucleic acid sequence is performed under low-stringency conditions.

24. A nucleic acid molecule identified by the method of claim 22.

25. A desaturase encoded by the nucleic acid molecule of claim 24.

26. A specific binding agent that binds the desaturase of claim 25.

27. The method of claim 22, wherein step (a) occurs in a PCR reaction.

28. The method of claim 22, wherein step (a) occurs during a library screening.

29. A method for creating a double bond between two carbons in a fatty acid, comprising:
contacting a fatty acid with a purified desaturase protein according to claim 1; and
allowing the desaturase protein to form a double-bond between two carbons in the fatty acid.

30. The method of claim 29, wherein the desaturase protein is expressed in a transgenic organism and the double-bond formation occurs in vivo.

31. The method of claim 30, wherein the desaturase is expressed in an organism selected from the group consisting of eukaryotes and prokaryotes.

32. The method of claim 29, wherein the desaturase is expressed in vitro, and the double-bond formation occurs in vitro.
